(19) United States
(12) Patent Application Publication
(10) Pub. No.: US 2004/0147545 A1
(43) Pub. Date: Jul. 29, 2004

(54) Derivatives of oxazolidinones as antibacterial agents

(76) Inventors: Marisabel Mourelle Mancini, Barcelona (ES); Juan Carlos Del Castillo Nieto, Barcelona (ES); Jose Hidalgo Rodriguez, Badalona (ES); Juan Huguet Clotet, Sant Joan Despi (ES)

Correspondence Address: STEINBERG & RASKIN, P.C., 1140 AVENUE OF THE AMERICAS, 15th FLOOR, NEW YORK, NY, 10036-5803, US

(21) Appl. No.: 10/469,283

(22) PCT Filed: Jun. 24, 2002
(86) PCT No.: PCT/IB02/02408

(30) Foreign Application Priority Data
Jun. 27, 2001 (ES) P 0101559

(51) Int. Cl. C07D 471/02; C07D 413/02; A61K 31/4745; A61K 31/4709

(52) U.S. Cl. 514/300; 514/312; 546/123; 546/156

(57) ABSTRACT

This invention discloses new fluorquinolonic derivatives of oxazolidinones of general formula (I) and processes for obtaining them, the corresponding pharmaceutical compositions and use thereof for manufacturing a medicament for the treatment of microbial infections. These new compounds are useful as antibacterial agents. Formula (I). Furthermore phenalen-type compounds according to general formula (II) are disclosed. Formula (II).

### FIELD OF THE INVENTION

This invention relates to fluorquinolonic derivatives of oxazolidinones. The compounds are useful as antibacterial agents.

### BACKGROUND OF THE INVENTION

For some years now the pharmaceutical industry has not been pursuing the development of new antibacterial agents specifically directed at gram-positive bacteria such as Staphylococci, Enterococci, Streptococci and mycobacteria. The gram-positive bacteria have nevertheless taken on particular importance due to the fact that they have developed resistance at an alarming rate to the conventionally used antibiotics, thus becoming organisms difficult both to treat and to eradicate from hospital environments. Examples of such strains are the Staphylococcus resistant to meticillin (MRSA), Enterococcus resistant to vancomycin (VRE), Staphylococcus epidermidis resistant to meticillin (MRSE), Staphylococcus pneumoniae resistant to penicillin (PRSP), etc.

The oxazolidinonic antibacterial agents are the most recent class of synthetic drugs which show high activity against gram-positive organisms. Owing to their new action mechanism, these compounds are effective against both sensitive and resistant pathogens, including MRSA, MRSE and VRE.

Various antibacterial oxazolidinones have been described in the patent literature, for example, to cite some of them, in WO 9507271. WO 9323384, WO 9854161. WO 9514684, WO 9721708, WO 9514684, WO 9730981. WO 9737980. WO 9801447, WO 9912914, WO 9613502.

All these patents describe the oxazolidinones as compounds active against resistant gram-positive organisms.

Owing to the constant appearance of new resistances, even to recently used antibiotics, it is desirable to develop powerful new antibiotics active against the resistant strains, preferably with a broad antimicrobial spectrum.

This invention provides new derivatives of oxazolidinones, with a broad antimicrobial spectrum due to their being active against gram-negative organisms while having improved activity against gram-positive organisms.

### DESCRIPTION OF THE INVENTION

The object of this invention are new fluorquinolonic derivatives of oxazolidinones of general formula (I):

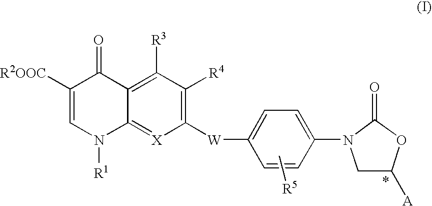

in which:

X: CR⁶ or N;

R¹: alkyl C₁-C₄, cycloalkyl C₃-C₆, alkenyl C₂-C₄, 2-hydroxyethyl, 2-fluoroethyl, or phenyl optionally substituted by 1 or 2 atoms of fluorine;

R²: H, alkyl C₁-C₄ or phenyl;

R³: H, halogen, alkyl C₁-C₄, or alkoxy C₁-C₄, amino;

R⁴: H or halogen;

R⁶: H, halogen, alkyl C₁-C₄, haloalkoxy C₁-C₄, or else R¹ and R⁶ together form a bridge of structure

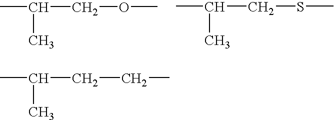

R⁵: H, halogen, OCH₃, alkoxy C₁-C₄, alkyl C₁-C₄, or haloalkyl C₁-C₄;

A: —CH₂—NH—R⁷, —CHOH—C≡CH

in which

R⁷: isoxazol, —CO—R⁸, —CS—R⁸, —CS—OR⁸, —COOR⁸, —CONHR⁸, —CSNHR⁸, —SO₂—R⁸ or

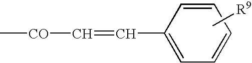

in which

R⁸: alkyl C₁-C₄, haloalkyl C₁-C₄, alkenyl C₂-C₄, aryl, alkyl C₁-C₄ substituted by an alkoxy group C₁-C₄, carboxyalkyl C₁-C₄, cyano, or amino, . . .

R⁹: H, alkyl C₁-C₄, alkenyl C₂-C₄, OH, alkoxy C₁-C₄, NR¹²R¹³, NO₂, halogen, or CO—R¹²;

R¹² and R¹³: independently, H or alkyl C₁-C₄;

W:

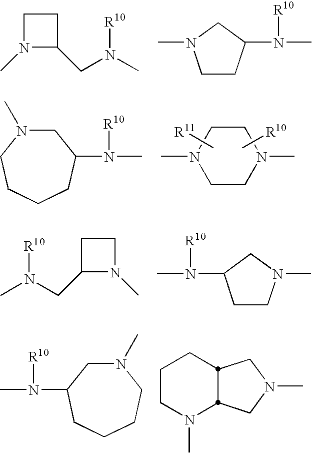

in which

R¹⁰ and R¹¹ are independently H, or alkyl C₁-C₄;

a pharmaceutically acceptable salt or solvate, or any geometric isomer, optical isomer or mixture of isomers thereof in any proportion or polymorph thereof.

Preferably, R¹ is cyclopropyl, ethyl, 2-fluoroethyl, phenyl or difluorophenyl, or else R¹ and R⁶ together form a bridge of structure:

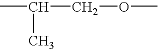

Preferably, R⁶ is H, CH₃, OCH₃, OCHF₂, F or Cl. More preferably, R⁶ is H or F.

Preferably, R⁴ is F or Cl and R³ is H.

Preferably, W is

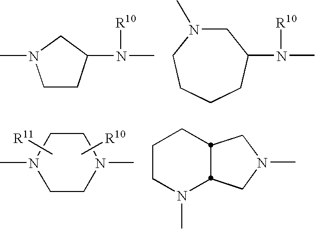

in which R¹⁰ and R¹¹ are as defined previously.

The compounds of the invention have a chiral centre in position C5 of the oxazolidinone ring. The preferred configuration of the C5 of the oxazolidinone ring is (S) for the compounds of formula (I) in which A=—CH₂—NH—R⁷ and (R) for the compounds of formula (I) in which A=—CHOH—C≡CH, in accordance with the Cahn-Ingold-Prelog nomenclature system.

Moreover, the compounds of formula (I) can contain other chiral centres. It is understood that the invention includes such optical isomers and diastereoisomers and mixtures thereof that possess antibacterial activity in any proportion.

The preferable compounds are selected from one of the following:

7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid

7-[3-({4-[5-(S)-(acetylamino-methyl)-2-oxo-oxazolidin-3-yl]-2-fluoro-phenyl}-methyl-amino)-azepan-1-yl]-1-cyclopropyl-6-fluoro-4-oxo-1,4-dihydro-quinoline-3-carboxylic acid

7-(4-{4-[5-(S)-(acetylamino-methyl)-2-oxo-oxazolidin-3-yl]-2-fluoro-phenyl}-piperazin-1-yl)-1-ethyl-6,8-difluoro-4-oxo-1,4-dihydro-quinoline-3-carboxylic acid

7-(4-{4-[5-(S)-(acetylamino-methyl)-2-oxo-oxazolidin-3-yl]-2-fluoro-phenyl}-piperazin-1-yl)-1-ethyl-6-fluoro-4-oxo-1,4-dihydro-quinoline-3-carboxylic acid

9-(4-{4-[5-(S)-(acetylamino-methyl)-2-oxo-oxazolidin-3-yl]-phenyl}-piperazin-1-yl)-8-fluoro-3-methyl-6-oxo-2,3-dihydro-6H-1-oxa-3a-aza-phenalen-5-carboxylic acid

9-[3-({4-[5-(S)-(acetylamino-methyl)-2-oxo-oxazolidin-3-yl]-2-fluoro-phenyl}-methyl-amino)-pyrrolidin-1-yl]-8-fluoro-3-methyl-6-oxo-2,3-dihydro-6H-1-oxa-3a-aza-phenalen-5-carboxylic acid

9-(4-{4-[5-(S)-(acetylamino-methyl)-2-oxo-oxazolidin-3-yl]-2-fluoro-phenyl}-piperazin-1-yl)-8-fluoro-3-methyl-6-oxo-2,3-dihydro-6H-1-oxa-3a-aza-phenalen-5-carboxylic acid

1-cyclopropyl-6-fluoro-7-[4-(2-fluoro-4-{5-(S)-[(3-methyl-thioureido)-methyl]-2-oxo-oxazolidin-3-yl}-phenyl)-piperazin-1-yl]-4-oxo-1,4-dihydro-quinoline-3-carboxylic acid

1-cyclopropyl-7-[4-(4-{5-(S)-[(3-ethyl-ureido)-methyl]-2-oxo-oxazolidin-3-yl}-2-fluoro-phenyl)-piperazin-1-yl]-6-fluoro-4-oxo-1,4-dihydro-quinoline-3-carboxylic acid

1-cyclopropyl-7-(4-{4-[5-(S)-(ethoxycarbonylamino-methyl)-2-oxo-oxazolidin-3-yl]-2-fluoro-phenyl}-piperazin-1-yl)-6-fluoro-4-oxo-1,4-dihydro-quinoline-3-carboxylic acid

1-cyclopropyl-6-fluoro-7-{4-[2-fluoro-4-(5-(S)-{[3-(4-fluoro-phenyl)-acryloylamino]-methyl}-2-oxo-oxazolidin-3-yl)-phenyl]-piperazin-1-yl}-4-oxo-1,4-dihydro-quinoline-3-carboxylic acid

1-cyclopropyl-7-[4-(4-{5-(S)-[(3-ethyl-thioureido)-methyl]-2-oxo-oxazolidin-3-yl}-2-fluoro-phenyl)-piperazin-1-yl]-6-fluoro-4-oxo-1,4-dihydro-quinoline-3-carboxylic acid

1-(2,4-difluoro-phenyl)-6-fluoro-7-(4-{2-fluoro-5-[5-(R)-(1-(R,S)-hydroxy-prop-2-inyl)-2-oxo-oxazolidin-3-yl]-phenyl}piperazin-1-yl)-4-oxo-1,4-dihydro-[1,8]naphthyridine-3-carboxylic acid ethyl ester

7-(4-{4-[5-(S)-(acetylamino-methyl)-2-oxo-oxazolidin-3-yl]-2-fluoro-phenyl}-piperazin-1-yl)-1-(2,4-difluoro-phenyl)-6-fluoro-4-oxo-1,4-dihydro-[1,8]naphthyridine-3-carboxylic acid ethyl ester

7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-[1,8]naphthyridine-3-carboxylic acid ethyl ester

7-(4-{4-[5-(S)-(acetylamino-methyl)-2-oxo-oxazolidin-3-yl]-2-fluoro-phenyl}-piperazin-1-yl)-6,8-difluoro-1-(2-fluoro-ethyl)-4-oxo-1,4-dihydro-quinoline-3-carboxylic acid ethyl ester

1-(2,4-Difluoro-phenyl)-6-fluoro-7-(4-{2-fluoro-4-[5-(S)-(isoxazol-3-ylaminomethyl)-2-oxo-oxazolidin-3-yl]-phenyl}-piperazin-1-yl)-4-oxo-1,4-dihydro-[1,8]naphthyridine-3-carboxylic acid ethyl ester

1-(2,4-difluoro-phenyl)-6-fluoro-7-(4-{2-fluoro-4-[5-(R)-(1-hydroxy-prop-2-inyl)-2-oxo-oxazolidin-3-yl]-phenyl}-piperazin-1-yl)-4-oxo-1,4-dihydro-[1,8]naphthyridine-3-carboxylic acid

7-(4-{4-[5-(S)-(acetylamino-methyl)-2-oxo-oxazolidin-3-yl]-2-fluoro-phenyl}-piperazin-1-yl)-1-(2,4-difluoro-phenyl)-6-fluoro-4-oxo-1,4-dihydro-[1,8]naphthyridine-3-carboxylic acid

7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-[1,8]naphthyridine-3-carboxylic acid

7-(4-{4-[5-(S)-(acetylamino-methyl)-2-oxo-oxazolidin-3-yl]-2-fluoro-phenyl}-piperazin-1-yl)-6,8-difluoro-1-(2-fluoro-ethyl)-4-oxo-1,4-dihydro-quinoline-3-carboxylic acid

1-(2,4-Difluoro-phenyl)-6-fluoro-7-(4-{2-fluoro-4-[5-(S)-(isoxazol-3-ylaminomethyl)-2-oxo-oxazolidin-3-yl]-phenyl}-piperazin-1-yl)-4-oxo-1,4-dihydro-[1,8]naphthyridine-3-carboxylic acid

1-ethyl-6,8-difluoro-7-[4-(2-fluoro-4-{5-[(3-methyl-thioureido)-methyl]-2-oxo-oxazolidin-3-yl}-phenyl)-piperazin-1-yl]-4-oxo-1,4-dihydro-quinoline-3-carboxylic acid

1-cyclopropyl-6-fluoro-7-[4-(2-fluoro-4-{2-oxo-5-(S)-[(3-propyl-thioureido)-methyl]-oxazolidin-3-yl}-phenyl)-piperazin-1-yl]-4-oxo-1,4-dihydro-quinoline-3-carboxylic acid

1-cyclopropyl-6-fluoro-7-[4-{2-fluoro-4-[5-(S)-(methanesulfonylamino-methyl)-2-oxo-oxazolidin-3-yl]-phenyl}-piperazin-1-yl)-4-oxo-1,4-dihydro-quinoline-3-carboxylic acid

7-(4-{4-[5-(S)-(Acetylamino-methyl)-2-oxo-oxazolidin-3-yl]-phenyl}-piperazin-1-yl)-1-ethyl-6,8-fluoro-4-oxo-1,4-dihydro-quinoline-3-carboxylic acid ethyl ester

1-cyclopropyl-6-fluoro-7-[4-(2-fluoro-4-{2-oxo-5-(S)-[(2,2,2-trifluoro-acetylamino)-methyl]-oxazolidin-3-yl}-phenyl)-piperazin-1-yl]-4-oxo-1,4-dihydro-quinoline-3-carboxylic acid

7-(4-{4-[5-(S)-(benzoylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid.

7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid methyl ester

7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid ethyl ester

7-(4-{4-[5-(S)-(acetylamino-methyl)-2-oxo-oxazolidin-3-yl]-2-fluoro-phenyl}-piperazin-1-yl)-1-ethyl-6,8-difluoro-4-oxo-1,4-dihydro-quinoline-3-carboxylic acid methyl ester

7-(4-{4-[5-(S)-(acetylamino-methyl)-2-oxo-oxazolidin-3-yl]-2-fluoro-phenyl}-piperazin-1-yl)-1-ethyl-6,8-difluoro-4-oxo-1,4-dihydro-quinoline-3-carboxylic acid ethyl ester

7-(4-{4-[5-(S)-(acetylamino-methyl)-2-oxo-oxazolidin-3-yl]-2-fluoro-phenyl}-piperazin-1-yl)-1-ethyl-6-fluoro-4-oxo-1,4-dihydro-quinoline-3-carboxylic acid methyl ester

7-(4-{4-[5-(S)-(acetylamino-methyl)-2-oxo-oxazolidin-3-yl]-2-fluoro-phenyl}-piperazin-1-yl)-1-ethyl-6-fluoro-4-oxo-1,4-dihydro-quinoline-3-carboxylic acid ethyl ester

9-(4-{4-[5-(S)-(acetylamino-methyl)-2-oxo-oxazolidin-3-yl]-phenyl}-piperazin-1-yl)-8-fluoro-3-methyl-6-oxo-2,3-dihydro-6H-1-oxa-3a-aza-phenalen-5-carboxylic acid methyl ester

9-(4-{4-[5-(S)-(acetylamino-methyl)-2-oxo-oxazolidin-3-yl]-phenyl}-piperazin-1-yl)-8-fluoro-3-methyl-6-oxo-2,3-dihydro-6H-1-oxa-3a-aza-phenalen-5-carboxylic acid ethyl ester

9-[3-({4-[5-(S)-(acetylamino-methyl)-2-oxo-oxazolidin-3-yl)-2-fluoro-phenyl}-methyl-amino)-pyrrolidin-1-yl]-8-fluoro-3-methyl-6-oxo-2,3-dihydro-6H-1-oxa-3a-aza-phenalen-5-carboxylic acid methyl ester

9-[3-({4-(5-(S)-(acetylamino-methyl)-2-oxo-oxazolidin-3-yl]-2-fluoro-phenyl}-methyl-amino)-pyrrolidin-1-yl]-8-5 fluoro-3-methyl-6-oxo-2,3-dihydro-6H-1-oxa-3a-aza-phenalen--carboxylic acid ethyl ester

9-(4-{4-[5-(S)-(acetylamino-methyl)-2-oxo-oxazolidin-3-yl]-2-fluoro-phenyl}-piperazin-1-yl)-8-fluoro-3-methyl-6-oxo-2,3-dihydro-6H-1-oxa-3a-aza-phenalen-5-carboxylic acid methyl ester

9-(4-{4-[5-(S)-(acetylamino-methyl)-2-oxo-oxazolidin-3-yl]-2-fluoro-phenyl}-piperazin-1-yl)-8-fluoro-3-methyl-6-oxo-2,3-dihydro-6H-1-oxa-3a-aza-phenalen-5-carboxylic acid ethyl ester

7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid methyl ester

7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid ethyl ester

1-cyclopropyl-6-fluoro-7-[4-(2-fluoro-4-{5-(S)-[(3-methyl-thioureido)-methyl]-2-oxo-oxazolidin-3-yl}-phenyl)-piperazin-1-yl]-4-oxo-1,4-dihydro-quinoline-3-carboxylic acid methyl ester

1-cyclopropyl-6-fluoro-7-[4-(2-fluoro-4-{5-(S)-[(3-methyl-thioureido)-methyl]-2-oxo-oxazolidin-3-yl}-phenyl)-piperazin-1-yl]-4-oxo-1,4-dihydro-quinoline-3-carboxylic acid ethyl ester

7-(4-{4-[5-(S)-(acetylamino-methyl)-2-oxo-oxazolidin-3-yl]-2-fluoro-phenyl}-piperazin-1-yl) -1-cyclopropyl-6-fluoro-4-oxo-1,4-dihydro-[1,8]naphthyridine-3-carboxylic acid methyl ester

7-(4-{4-[5-(S)-(acetylamino-methyl)-2-oxo-oxazolidin-3-yl]-2-fluoro-phenyl}-piperazin-1-yl)-6,8-difluoro-1-(2-fluoro-ethyl)-4-oxo-1,4-dihydro-quinoline-3-carboxylic acid methyl ester

1-Ethyl-6,8-difluoro-7-[4-(2-fluoro-4-{5-(S)-[(3-methyl-thioureido)-methyl]-2-oxo-oxazolidin-3-yl)-phenyl)-piperazin-1-yl]-4-oxo-1,4-dihydro-quinoline-3-carboxylic acid ethyl ester

In this invention the term “a pharmaceutically acceptable solvate” is taken to mean a hydrate or solvate of an alcohol C₁-C₄.

In this invention, the term “pharmacologically acceptable salts” includes salts of alkaline metals such as sodium or potassium and salts of alkaline earth metals such as calcium or magnesium, as well as acid-addition salts formed with inorganic and organic acids such hydrochlorides, hydrobromides, sulphates, nitrates, phosphates, formiates, mesylates, citrates, benzoates, fumarates, maleates, lactates and succinates, among others.

The pharmacologically acceptable salts are prepared by reaction of a compound of formula (I) with a suitable quantity of a base such as sodium, potassium, calcium or magnesium hydroxyde, or sodium methoxide, sodium hydride, potassium tert-butoxyde and the like in solvents such as ether, THF, methanol, ethanol, tert-butanol, isopropanol, dioxane, etc., or else in a mixture of solvents. The addition salts, where applicable, can be prepared by treatment with acids, such as hydrochloric, hydrobromic, sulphuric, nitric, phosphoric, formic, methanesulphonic, citric, benzoic, fumaric, maleic, lactic and succinic, in solvents such as ether, alcohols, acetone, THF, ethyl acetate, or mixtures of solvents.

The stereoisomers of this invention can be prepared by using reagents in a single enantiomeric form in processes where this is possible or by carrying out the reaction in the presence of reagents or catalysts in their single enantiomeric form or by resolution of mixtures of stereoisomers by conventional methods. Some of the preferred methods include resolution of diastereoisomeric salts formed with chiral acids such as mandelic, camphorsulphonic, tartaric acid and the like. Methods commonly used are included in Jaques et al. in “Enantiomers, Racemates and Resolution” (Wiley Interscience, 1981).

In the definitions of this invention, an alkyl group C₁-C₄, as a group or as part of a group, is taken to mean a lineal or branching alkyl group which contains up to 4 atoms of carbon. Thus it includes, for example, methyl, ethyl, propyl, isopropyl, butyl, isobutyl, sec-butyl and tert-butyl.

Likewise, an alkoxy group C₁-C₄ includes, for example, a methoxy, ethoxy, propoxy, isopropoxy, butoxy, isobutoxy, sec-butoxy and tert-butoxy group.

An alkenyl group C₂-C₄ includes, for example, a vinyl, alyl, propenyl and 1-butenyl, 2-butenyl and 3-butenyl group.

A haloalkyl group C₁-C₄ means an alkyl group C₁-C₄ substituted by one or more atoms of halogen, the same or different. It thus includes, for example, chloromethyl, fluoromethyl, trifluoromethyl, chloroethyl, fluoroethyl, difluoroethyl, trifluoroethyl, fluoropropyl, chloropropyl, etc.

A haloalkoxy group C₁-C₄ means an alkoxy group C₁-C₄ substituted by one or more atoms of halogen, the same or different. Thus it includes, for example, chloromethoxy, fluoromethoxy, trifluoromethoxy, chloroethoxy, fluoroethoxy, difluoroethoxy, trifluoroethoxy, fluoropropoxy, chloropropoxy, etc.

A cycloalkyl group C₃-C₆ represents a cyclopropyl, cyclobutyl, cyclopentyl and cyclohexyl group.

The term halogen, in this invention, refers to F, Cl, Br, I, preferably F and Cl.

The term aryl, in this invention, includes phenyl and naphthyl optionally substituted by up to five substituents, the same or different, preferably up to two, in any position of the ring. Suitable substituents include halogen, amino, hydroxy, alkyl C₁-C₄, alkoxy C₁-C₄, phenyl.

The compounds of this invention can be prepared in various ways. They can be prepared by using the methods described below, together with methods known in the field of organic chemical synthesis, or by the variations that might be made thereto by an expert in the subject. Preferred methods include, but are not limited to, those described below. The reactions are carried out in the solvents appropriate for the reagents and materials used and suited for the transformations carried out. An expert in organic synthesis will understand that the functional groups present in the molecule must be consistent with the proposed transformations. This may in some cases require modifying the order of the synthesis steps or selecting one particular method rather than another, in order to obtain the desired compound of the invention. Moreover, in some of the procedures described below it may be desirable or necessary to protect the reagent functional groups present in the compounds or intermediates of this invention with conventional protecting groups. Various protecting groups and procedures for introducing them and removing them are described in Greene and Wuts (Protective Groups in Organic Synthesis, Wiley and Sons, 1999). All the references cited herein are incorporated integrally by reference.

The compounds of formula (I) can be obtained by reaction of a compound of formula (II), with a compound of formula (III):

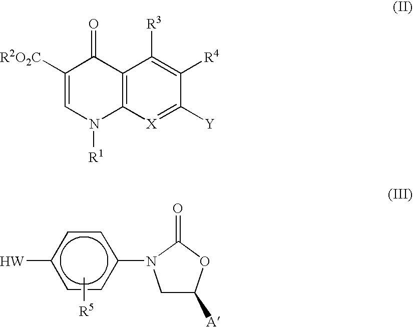

in which

A′ is:

a) —CH₂—NH—R⁷

b) —CHOH—C≡CH

c)

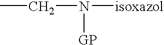

Y is an leaving group, such as an atom of halogen (F, Cl, Br, I), a tosilate or mesylate group and the like;

R^{1.} R², R³, R⁴, R⁵, X and W have the meaning defined above;

GP is an amine protecting group.

Alternatively, the compounds of formula (I) in which A=—CHOH—C≡CH can also be obtained by reaction of a compound of formula (IV) with 2,3-hydroxy-pent-4-inyl p-toluenesulphonate:

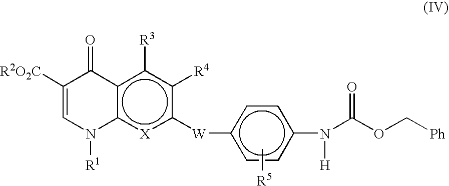

in which R^{1.} R², R³, R⁴, R⁵, X and W have the meaning defined above.

The compounds of formula (I) in which A=—CH₂—NH—R⁷ and R⁷ is different from isoxazol, can also be obtained by reaction of a compound of formula (V):

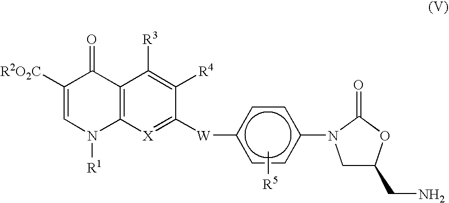

in which R^{1.} R², R³, R⁴, R⁵, X and W have the meaning defined above, with a compound of formula (VI) or with a compound of formula (VII)

R⁷-L (VI)

R⁸—N═C═Z (VII)

in which

L is a good leaving group, such as an atom of halogen (F, Cl, Br, I), a tosilate or mesylate group and the like;

Z is Oxygen or Sulphur, and

R⁷ and R⁸ have the meaning defined above, with R⁷ being different from isoxazol.

The compounds of formula (I) in which A=—CH₂—NH—R⁷ and R⁷ is isoxazol can also be obtained by reaction of a compound of formula (VIII)

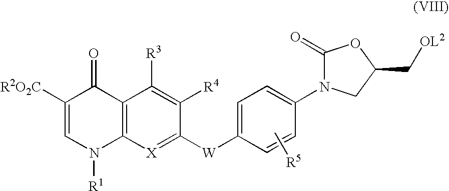

in which

OL² represents a good leaving group, such as a residue of aryl or methyl sulphonic acid, whether substituted or not substituted, preferably by a tosilate or mesylate group;

R^{1.} R², R³, R⁴, R⁵, X and W have the meaning defined above;

with isoxazolil-3-amine, with the amino group suitably protected with an amine protecting group, for example with Troc (2,2,2-trichloroethoxycarbonyl).

The compounds of formula (I), in which R²═H can also be obtained by hydrolysis of a boron chelate of formula (IX):

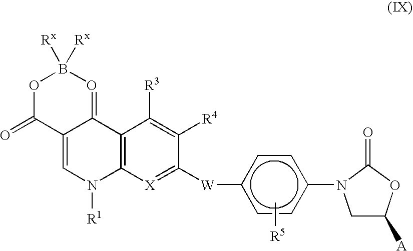

in which

R^{x} can be F or CH₃COO—;

A, R^{1.} R³, R⁴, R⁵, X and W have the meaning defined above.

And if required, after any of the methods described herein, one or more of the following optional steps can be carried out:

Converting a compound of general formula (I) into another compound of general formula (I);

Eliminating any protecting group;

Preparing a pharmacologically acceptable salt of a compound of formula (I) and/or pharmacologically acceptable solvate thereof.

The reaction of the compounds of formula (II) with compounds of formula (III) is carried out in an organic solvent in the presence of an organic base. Preferably the reaction is carried out in solvents such as pyridine, acetonitrile, dimethylformamide, N-methylpyrrolidone, etc. in the presence of bases such as triethylamine, DBU, diisopropylethylamine, etc.

The reaction of compounds of formula (IV) with 2,3-hydroxy-pent-4-inyl p-toluenesulphonate is carried out in an aprotic solvent such as N,N-dimethylformamide, THF, preferably THF, at low temperature, preferably at −68° C., and in the presence of a base such as n-butyllithium, lithium tert-butoxide, LDA, preferably in n-butyllithium.

The reaction of compounds of formula (V) with a compound of formula (VI) is carried out in an organic aprotic solvent such as acetonitrile, dichloromethane or pyridine or in a mixture of an organic solvent and water in the presence of a base. Preferably L is Cl, EtO, etc, so that R⁷-L can be an acid, an acid chloride, an anhydride, an ester, a dithioester, an alkyl or aryl chloroformiate, etc. The reaction of compounds of formula (V) with a compound of formula (VII) is preferably carried out in pyridine.

The reaction of the compounds of formula (VIII) with isoxazolil-3-amine, with the amino group suitably protected, is carried out in an aprotic solvent such as N,N-dimethylformamide, N,N-dimethylacetamide, preferably in N,N-dimethylformamide, at a temperature between 0 and 70° C., and in the presence of a strong base such as sodium hydride, lithium tert-butoxide, sodium tert-butoxide, potassium tert-butoxide or sodium amide, preferably sodium hydride.

Hydrolysis of the compounds of formula (IX) can be carried out according to the methods previously described in the literature (Masuhiro Fujita Chem. Pharm. Bull. (1988), 46(5), 787-796, Joseph P. Sánchez J. Med. Chem. (1995), 38, 4478-4487)

For R^{x}=F, the hydrolysis is carried out preferably in a mixture of alcohol-water in the presence of a base. As water-alcohol mixture it is preferable to use ethanol-water or methanol-water and as base it is preferable to use an organic base such as triethylamine or another secondary or tertiary amine such as tributylamine, diisopropylethylamine, DBU, etc. The reaction is carried out at a temperature that can range between room temperature and the reflux temperature of the water-alcohol mixture. The reaction is carried out preferably at the reflux temperature of the water-alcohol mixture.

When R^{x}=CH₃COO the hydrolysis is carried out preferably in a mixture of an organic aprotic solvent and another protic solvent in the presence of a base. As aprotic solvent it is preferable to use acetonitrile and as protic solvent it is preferable to use water. As base it is preferable to use an inorganic base such as sodium, lithium or potassium hydroxide or sodium, lithium or potassium carbonate, etc.

A reaction of interconversion of a compound of formula (I) into another compound of formula (I) consists, for example, in hydrolysing a compound of formula (I) in which R² is an alkyl C₁-C₄ or phenyl radical to convert it into a compound of formula I in which R² is hydrogen. The hydrolysis is carried out preferably in a water-alcohol medium preferably using as base an inorganic base. Still more preferably, the hydrolysis is carried out in ethanol-water or methanol-water, while sodium, lithium or potassium hidroxide is used as a base.

Another example of reaction of interconversion of a compound of formula (I) in another compound of formula (I) consists in the esterification of a compound of formula (I) in which R² is hydrogen, to yield another compound of formula (I) in which R² is an alkyl C₁-C₄ or phenyl radical, by the conventional methods of esterification described in the literature. For example, by reaction of a compound of formula R²—OH with the compound of formula (I) in which R² is hydrogen, having previously activated the carboxylic acid with carbonyl diimidazole, or else having previously converted the carboxylic acid into an acid chloride by reaction with thionyl chloride, or else having converted it into mixed anhydride by reaction with alkyl chloroformiate.

Also object of invention are the compounds of formula (V), (X) and (XI):

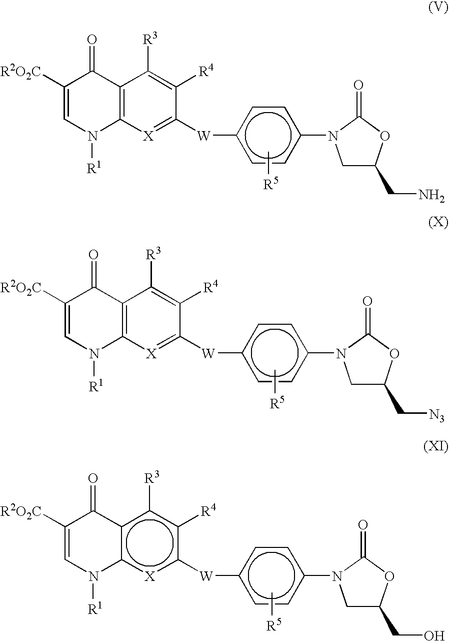

in which R^{1.} R², R³, R⁴, R⁵, X and W have the meaning defined above. These compounds are useful as intermediates for making the compounds of formula (I) of this invention.

Described below are some of the procedures for making the intermediates used for preparing the compounds of formula (I).

The compounds of formula (V), (X) and (XI) can be obtained in accordance with schemes 1A and 1B.

Thus, the compounds of formula (V) can be obtained:

a. by reaction of a compound of formula (II) or of formula (XII) with a compound of formula (XIII):

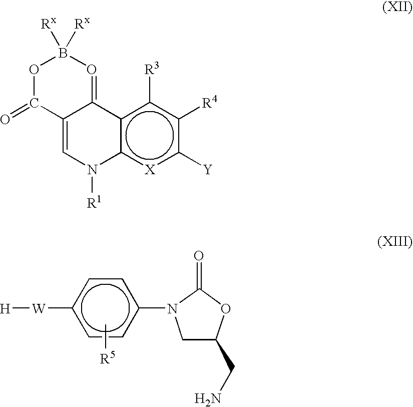

The reaction can be carried out under the conditions described above for the reaction of a compound of formula (II) with a compound of formula (III);

b. by catalytic reduction of a product of formula (X) or by reduction of the azide group chemically with triphenylphosphine, etc.

The compounds of formula (X) can in their turn be obtained:

a. by reaction of a compound of formula (XII) or of formula (II) with a compound of formula (XIV):

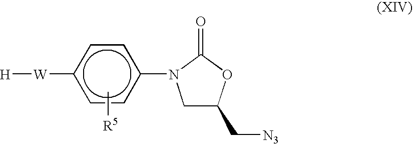

The reaction can be carried out under the conditions described above for the reaction of a compound of formula (II) with a compound of formula (III);

b. from a compound of formula (XI) by conversion of the hydroxyl group into a good leaving group, such as mesylate, tosilate or halogen and subsequent reaction with sodium azide.

The compounds of formula (XI) can in their turn be obtained:

a. by reaction of a compound of formula (XII) or of formula (II) with a compound of formula (XV):

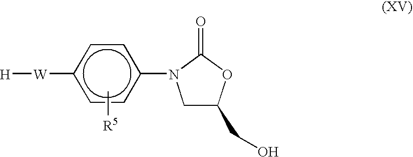

The reaction can be carried out under the conditions described above for the reaction of a compound of formula (II) with a compound of formula (III);

b. by reaction of a compound of formula (IV) with (R)-glycidil butirate. The reaction is carried out in an aprotic solvent such as N,N-dimethylformamide, THF, preferably THF, at low temperature, preferably at −68° C., and in the presence of a base such as n-butyllithium, lithium tert-butoxide, LDA, preferably in n-butyllithium.

Utilisation of the compounds of formula (XII) to obtain the three foregoing intermediates requires an additional step of hydrolysis of the boron chelate, as indicated in schemes 1A and 1B, which step is carried out under the conditions described above for hydrolysis of the compound of formula (IX).

The compounds of formula (VIII) can be obtained by reaction of a compound of formula (XI) with aryl or methyl sulphonyl chloride, substituted or not substituted, preferably with mesyl chloride or p-toluenesulphonyl chloride, in an aprotic solvent, such as methylene chloride, and in the presence of an organic base, such as triethylamine.

The compounds of formula (IX) can be obtained by reaction of a compound of formula (XII) with a compound of formula (III). The reaction can be carried out under the conditions described above for the reaction of a compound of formula (II) with a compound of formula (III).

The products of formula (II) and of formula (XII) are obtained according to the methods described in the literature. This products have been used as intermediates in the synthesis of quinolones and similar with antibacterial activity such as cyprofloxacin, ofloxacin, moxyfloxacin, norfloxacin, tosufloxacin, etc. (See patents WO 8807993, WO 8807998, WO 9006922, JP 59122470. JP 58029789, EP 0351889).

The compounds of formula (III), (XIII), (XIV) and (XV) can be obtained in accordance with scheme 2.

Thus, the compounds of formula (IIIa), (XIII) and (XIV) can be obtained from a compound of formula (XVI) by conversion of the hydroxyl group into an NH₂, N₃ or NHR⁷ group, in accordance with reactions well-known to an expert in organic chemistry.

The compounds of formula (IIIb) can be obtained by reaction of a compound of formula (XVII) with 2,3-hydroxy-pent-4-inyl p-toluenesulphonate, under conditions analogous to those described for the reaction of a compound of formula (IV) with said reagent.

The compounds of formula (IIIc) can be obtained by reaction of a compound of formula (XVI) with isoxazolil-3-amine, with the amino group suitably protected, for example with Troc, and prior conversion of the hydroxyl group into a good leaving group, for example, mesylate, tosilate, halogen, etc.

The compounds of formula (IV) can be obtained according to the following scheme:

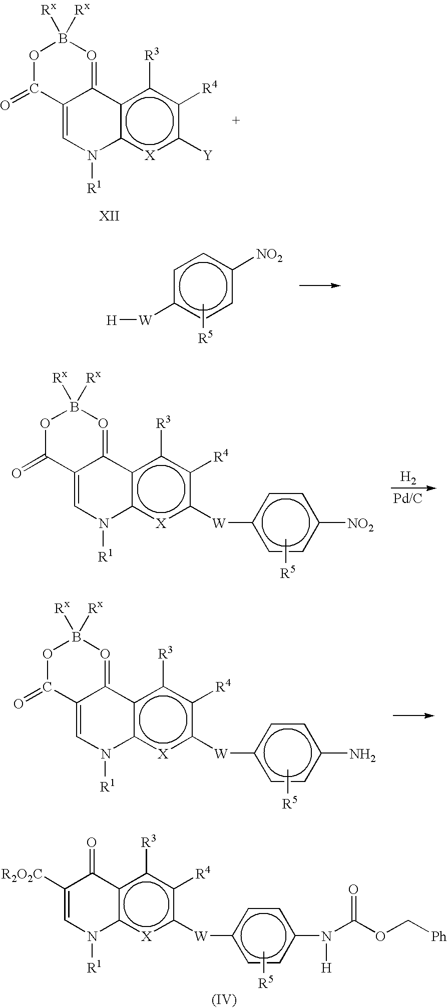

The reactions are carried out in suitable solvents, and under conventional conditions. The schemes indicate the preferred reaction conditions.

The 2,3-hydroxy-pent-4-inyl p-toluenesulphonate is obtained according to the procedure described in EP 1029854A1.

The compounds of formula (VI) and of formula (VII) are commercial, are extensively described in the literature or can be prepared by methods analogous to those known in the state of the art from products commercially available.

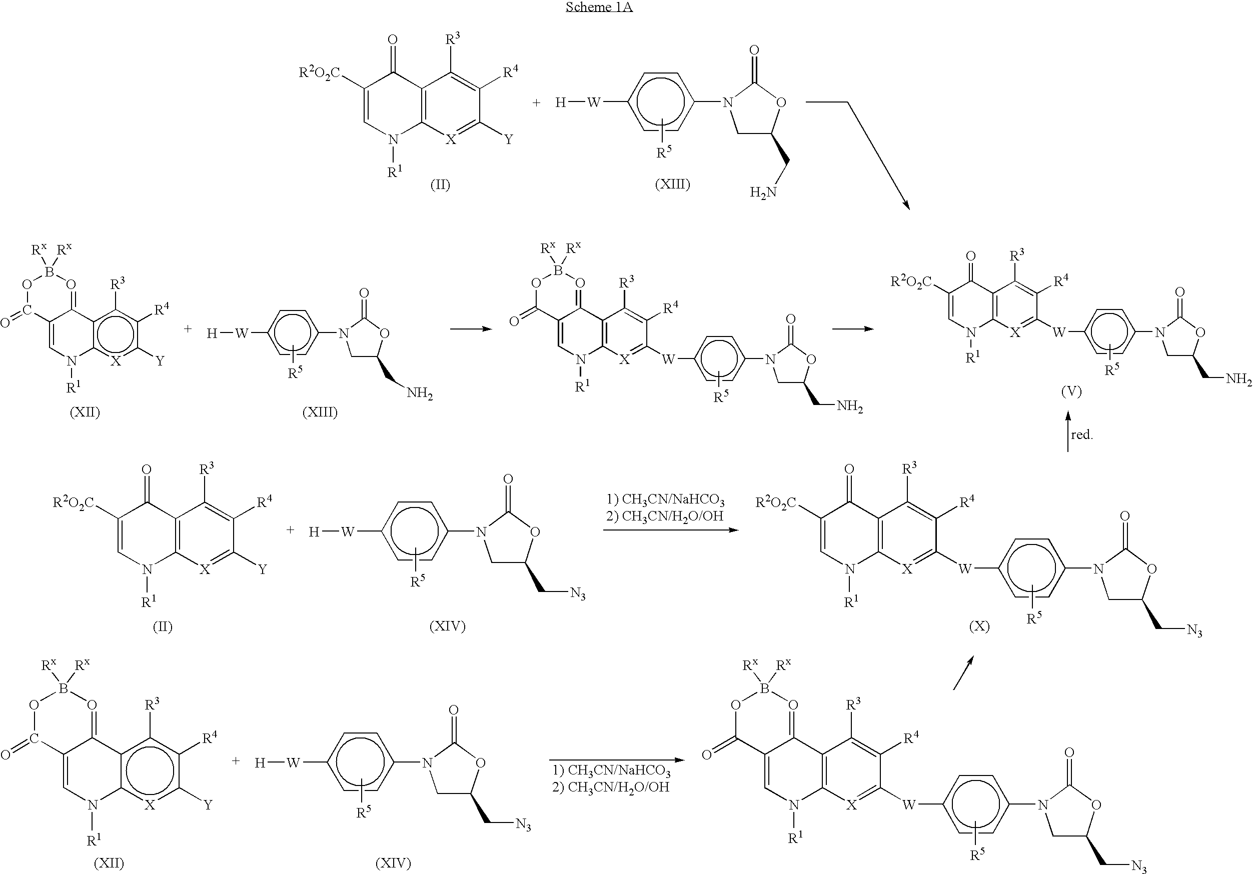

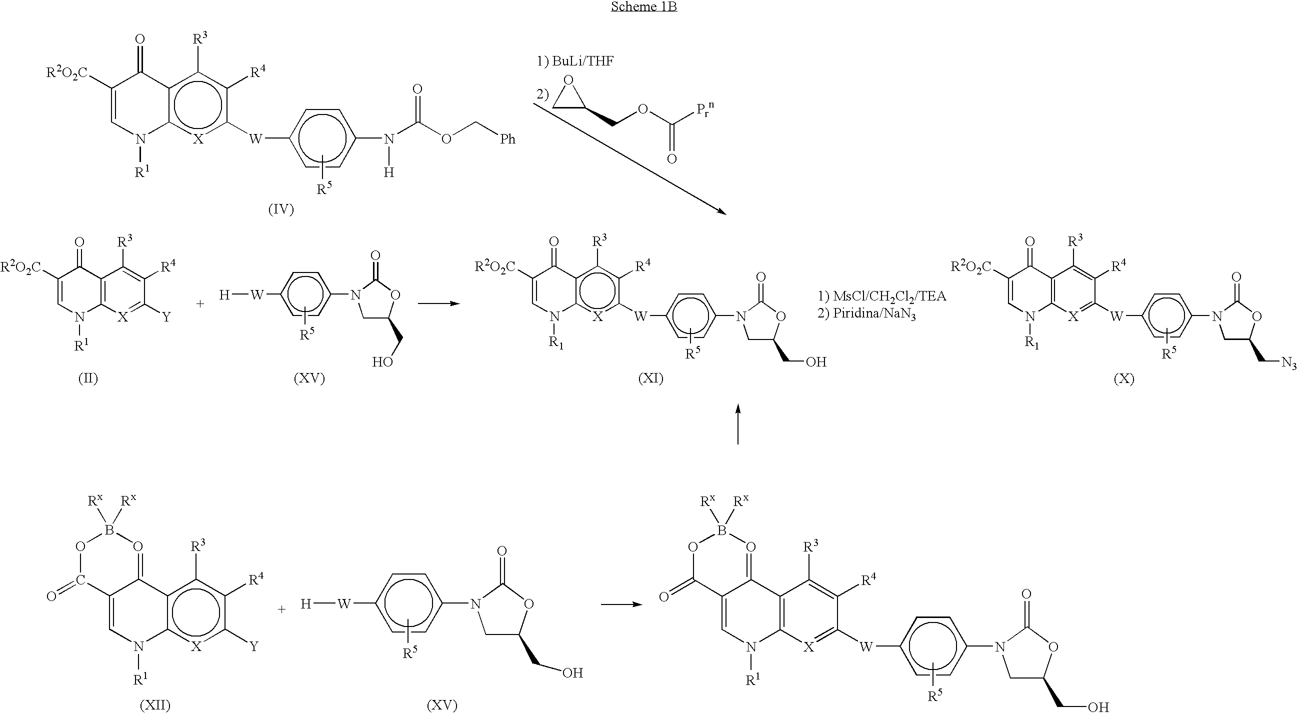

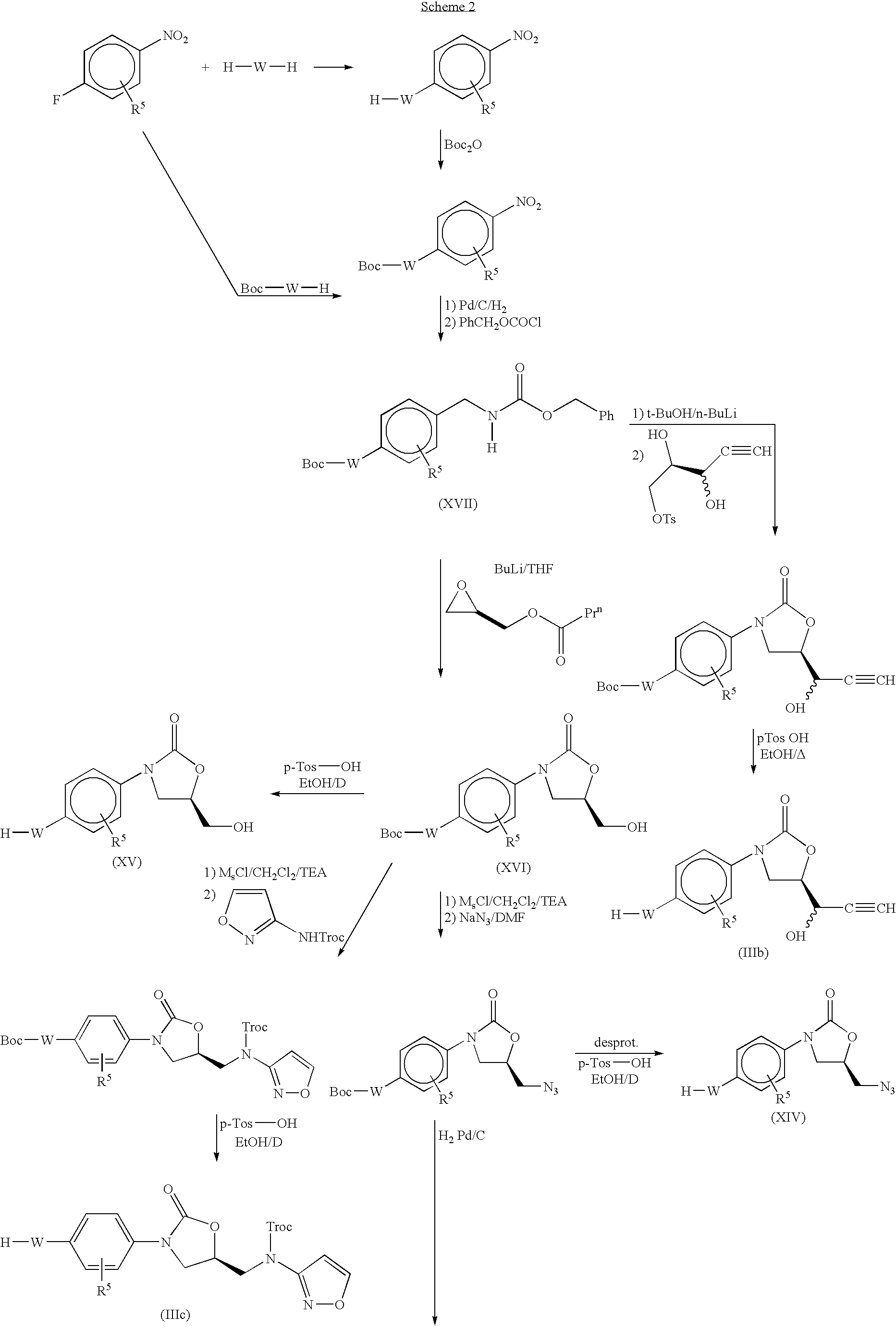

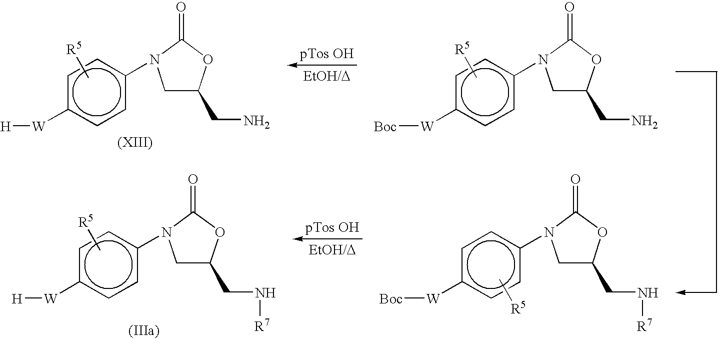

Also object of this invention are compositions which include a compound of general formula (I), a pharmaceutically acceptable salt or solvate, or any geometric isomer, optical isomer or mixture of isomers thereof in any proportion or polymorph thereof, in a therapeutically active quantity and a suitable quantity of at least one pharmacologically acceptable excipient.

The compositions of the invention can be formulated in solid or liquid form following the conventional pharmaceutical techniques. The solid formulations include tablets, capsules, sachets, powders, suppositories, etc. The excipients can include diluents, disintegrators, wetting agents, lubricants, colourants, flavourings or other conventional adjuvants. The typical solid excipients include, for example, microcrystalline cellulose, starch, polyvinylpyrrolidone, magnesium stearate or sodium lauryl sulphate. The liquid compositions include solutions, suspensions or emulsions. They can consist in solutions in water or in water-propyleneglycol or water-polyethylenglycol systems, also optionally containing flavourings, colourants, stabilisers and thickeners.

The compositions can be administered orally, parenterally or topically.

The compounds of formula (I) show activity as antibacterial agents. Advantageously they possess a broad spectrum of activity against gram-positive bacteria such as Staphylococcus, Streptococcus, Enterococcus and the like, as well as against gram-negative bacteria such as E. Coli, H. Influenzae, M. catarrahalis, etc., and even against strains resistant to known antibiotics such as meticillin, vancomicine, penicillin, etc. They are also active against anaerobic microorganisms such as Bacteroides fragilis. Also object of this invention, therefore, is the use of a compound of formula (I) for making a pharmaceutical composition for the treatment of microbial infections, in humans or warm-blooded animals.

Below, and by way of non-restrictive explanation of the invention, the following examples are set out.

### EXAMPLES OF SYNTHESIS

### Preparation of Intermediates

### Reference Example No. 1

### 1-Cyclopropyl-6-fluoro-7-[4-(2-fluoro-4-nitro-phenyl)-piperazin-1-yl]-4-oxo-1,4-dihydro-quinoline-3-carboxylic Acid Diacethoxyboron Chelate

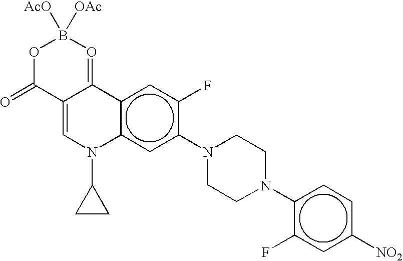

To 10 g (0.024 mol) of 1-cyclopropyl-7-chloro-6-fluoro-4-oxo-1,4-dihydro-quinoline-3-carboxylic acid diacethoxyboron chelate (obtained according to WO 8807998) in 150 ml of acetonitrile are added 5.4 g (0.024 mol) of 1-(2-fluoro-4-nitro-phenyl)piperazine (obtained according to the method described by S. J. Brickner and col. J. Med. Chem. 1996, 39, 673-679) and 2 g (0.024 moles) of sodium bicarbonate.

The reaction is heated to reflux for 48 h. It is concentrated to dryness and the residue is treated with 100 ml of water and extracted with 3×100 ml of dichloromethane. The organic phase is dried and concentrated and the residue is chromatographed on silica gel.

Elution with dichloromethane/ethanol 98/2 yields 6.7 g of the product of the title.

¹H-RMN: (CDCl3, 200 MHz, δppm)): 9.08 (s,1H); 8.14 (d, 1H); 8.10-7.94 (s.c., 2H); 7.56 (d, 1H); 7.01 (t,1H); 3.82-3.75 (m, 1H); 3.75-3.50 (s.c., 8H); 2.04 (s, 6H); 1.64-1.30 (s.c., 4H).

### Reference Example No. 2

### 7-[4-(4-amino-2-fluoro-phenyl)piperazin-1-yl]-1-cyclopropyl-6-fluoro-4-oxo-1,4-dihydro-quinoline-3-carboxylic Acid Diacethoxyboron Chelate

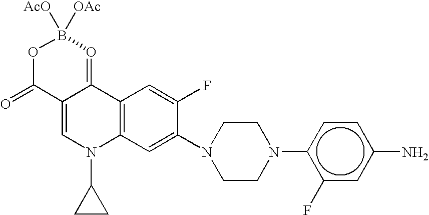

To 6.7 g (0.011 mol) of the product obtained in the previous example, in 50 ml of dimethylformamide, are added 0.7 g of 10% Pd/C paste and it is placed under hydrogen atmosphere at 40° C. and atmospheric pressure. When the reaction has finished it is filtered over decalite and the decalite washed with 20 ml of DMF.

The filtrate liquids are poured onto 700 ml of water and extracted with 3×200 ml of dichloromethane. The organic phase is concentrated to dryness and the residues chromatographed on silica gel.

Elution with dichloromethane-ethanol 95/5 yields 2.6 g of the product of the title as yellow solid.

¹H-RMN (CDCl3, 200 MHz, δ(ppm)): 9.04 (s, 1H); 8.10 (d, 1H); 7.45 (d, 1H); 6.84 (dd, 1H); 6.44-6.36 (s.c., 2H); 3.79-3.64 (m, 1H); 3.62-3.56 (s.c., 4H); 3.24-3.16 (s.c., 4H); 2.05 (s, 6H); 1.80-1.20 (s.a., 2H, NH₂); 1.58-1.24 (s.c., 4H).

### Reference Example No. 3

### 7-[4-(4-benzyloxycarbonylamino-2-fluoro-phenyl)-piperazin-1-yl]-1-cyclopropyl-6-fluoro-4-oxo-1,4-dihydro-quinoline-3-carboxylic Acid

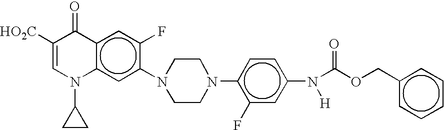

To 2.62 g (4.58 mmol) of the product obtained in the previous reference example in 30 ml of THF and 10 ml of water is added 0.4 g (5 mmol) of sodium bicarbonate. Onto the previous solution is added dropwise 0.8 g (5 mmol) of benzyl chloroformiate and is maintained with stirring for 48 h. It is concentrated to dryness, 50 ml of water are added and it is extracted with 3×75 ml of dichloromethane.

The organic phase is dried and concentrated. The residue is stirred with 10 ml of dichloromethane for 10 minutes and the precipitate obtained is filtered.

2 g of the product of the title are obtained thereby.

¹H-RMN (DMSO, 200 MHz, δ (ppm)): 9.84 (s.a., 1H); 8.64 (s, 1H); 7.92 (d, 1H); 7.61 (d, 1H); 7.50-7.30 (s.c., 6H); 7.22-7.01 (s.c., 2H); 5.18 (s, 2H); 3.92-3.78 (s.a., 1H); 3.70-3.10 (s.c., 8H); 1.42-1.10 (s.c., 4H).

### Reference Example No. 4

### 1-cyclopropyl-6-fluoro-7-{4-[2-fluoro-4-5-(R)-hydroxymethyl-2-oxo-oxazolidin-3-yl)-phenyl]-piperazin-1-yl}-4-oxo-1,4-dihydro-quinoline-3-carboxylic Acid

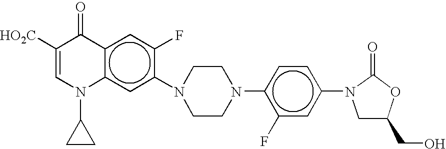

To 2.2 g (3.7 mmol) of the product obtained in the previous preparation in 60 ml of THF cooled to −78° C. is added dropwise 3 ml (7.14 mmol) of n-butyllithium 2.5 M in hexane.

The reaction is maintained at −78° C. for 1 h and then 0.51 g (3.57 mmol) of (R)-glycidil butirate dissolved in 10 ml of THF are added.

It is allowed to reach room temperature and stirred thus for 16 h.

20 ml of saturated solution of ammonium chloride is added and it is concentrated until the THF is removed. 50 ml of water are added and this is extracted with 3×100 ml of dichloromethane-ethanol (90/10).

The organic phase is dried and concentrated. The residue is chromatographed on silica gel. Elution with dichloromethane-ethanol (90/10) yields 0.5 g of the product of the title.

¹H-RMN (DMSO-d₆, 200 MHz, δ (ppm)): 8.70 (s, 1H); 7.96 (d, 1H); 7.70-7.36 (s.c., 3H); 7.30-7.10 (s.c., 2H); 5.20-5.10 (s.a., 1H); 4.8-4.64 (m, 1H); 4.20-4.04 (m, 1H); 3.92-3.14 (s.c., 11H); 1.43-1.16 (s.c., 4H).

### Reference Example No. 5

### 7-{4-[4-(5-(R)-azidomethyl-2-oxo-oxazolidin-3-yl)-2-fluoro-phenyl]-piperazin-1-yl}-1-cyclopropyl-6-fluoro-4-oxo-1,4-dihydro-quinoline-3-carboxylic Acid

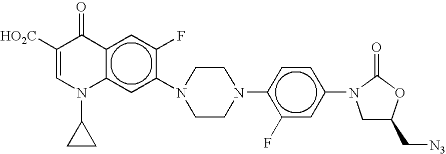

Method 1 :

To 0.5 g (0.92 mmol) of the product obtained in the previous preparation in 10 ml of dry dichloromethane is added 2.6 ml of triethylamine and it is then cooled to 0° C. 1.4 ml of methanesulphonyl chloride is added and this is then stirred at 0° C. for 1 h.

It is poured onto water-ice (30 ml/20 g) saturated with sodium bicarbonate and the organic phase is decanted. It is dried on sodium sulphate, filtered and concentrated.

To the residue is added 10 ml of dimethylformamide and 1.17 g of sodium azide. This is heated to 75° C. and stirred at this temperature for 16 h.

It is poured onto 100 ml of water and extracted with 3×100 ml of ethyl acetate. The organic phase is dried and concentrated and the residue is chromatographed on silica gel. Elution with dichloromethane-ethanol (90/10) yields 40 mg of the product of the title.

Method 2:

To 1.5 g (4.7 mmol) of 5-(R)-azidomethyl-3-(3-fluoro-4-piperazin-1-yl-phenyl)-oxazolidin-2-one (Reference Example No. 19) and 1.9 g (4.7 mmol) of acid 1-cyclopropyl-7-chloro-6-fluoro-4-oxo-1,4-dihydroquinoline-3-carboxylic acid diacethoxyboron chelate (obtained according to WO 8807998) in 60 ml of acetonitrile is added 0.4 g (4.7 mmol) of sodium bicarbonate and this is heated to reflux for 48 h.

It is concentrated to dryness and the residue is treated with 100 ml of water and extracted with 3×100 ml of CH₂Cl₂. The organic phase is dried and concentrated and the residue is chromatographed on silica gel.

Elution with CH₂Cl₂/EtOH 95/S yields 1.1 g of the product of the title as diacetoxiboron chelate.

The 1.1 g thus obtained is dissolved in a mixture of 28 ml of water, 28 ml of acetonitrile and 8 ml of sodium hidroxide 1N. This is stirred at room temperature for 3 h, the acetonitrile is concentrated and 8 ml of hydrochloric acid 1N is added.

The precipitated solid is filtered, yielding 0.6 g of product identical to that obtained by method 1.

¹H-RMN (CDCl₃, 200 MHz, δ (ppm)): 8.79 (s, 1H); 8.01 (d, 1H); 7.54-7.24 (s.c., 2H); 7.16-6.90 (s.c., 2H); 4.83-4.70 (m, 1H); 4.42-4.34 (m, 1H); 4.10-3.20 (s.c., 12H); 1.44-1.12 (s.c., 4H)

### Reference Example No. 6

### 3(R,S)-[(2-fluoro-4-nitro-phenyl)-methylamino]-pyrrolidine-1-carboxylic Acid Tert-butyl Ester

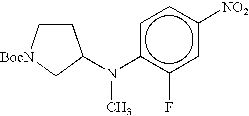

To 7 g (0.0375 mol) of 3(R,S)-methylamino-pyrrolidine-1-carboxylic acid tert-butyl ester and 4.11 ml (0.0375 mol) of 3,4-difluoronitrobenzene in 80 ml of DMF is added 3.15 g of sodium bicarbonate and this is heated at 45° C. for 16 h.

It is poured onto 800 ml of water and extracted with 3×300 ml of AcOEt. The organic phase is dried and concentrated and the residue is chromatographed on silica gel.

Elution with dichloromethane-ethanol 95/5 yields 7.9 g of the product of the title.

¹H-RMN (CDCl₃, 200 MHz, δ (ppm)): 8.00-7.88 (s.c, 2H); 6.88 (dd, 1H); 4.45-4.30 (m, 1H); 3.75-3.50 (s.a., 4H); 3.45-3.25 (s.c, 4H); 2.95 (s, 3H); 2.18-2.07 (m, 2H); 1.49 (s, 9H).

### Reference Example No. 7

### 3(R,S)-[(2-fluoro-4-nitro-phenyl)-methyl-amino]-azepan-1-carboxylic Acid Tert-butyl Ester

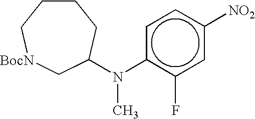

Following the previous procedure and using 3(R,S)-methylamino-azepan-1-carboxylic acid tert-butyl ester, the product of the title is obtained.

¹H-RMN (CDCl₃, 200 MHz, δ (ppm)): 8.10-7.80 (m, 2H); 6.90 (dt, 1H); 4.05-3.10 (m, 5H); 2.94 (m, 3H); 1.50 and 1.41 (s, 9H); 1.20-2.10 (m, 6H).

### Reference Example No. 8

### 4-(4-Benzyloxycarbonylamino-phenyl)-piperazin-1-carboxylic Acid Tert-butyl Ester

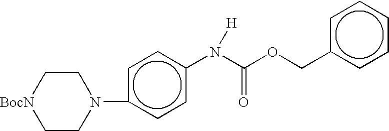

To 72.7 g (0.236 mol) of 4-(4-nitro-phenyl)-piperazin-1-carboxylic acid tert-butyl ester (WO 9725323), in 600 ml of THF and 125 ml of water is added 7.27 g of 10% Pd/C paste and it is placed under atmosphere of hydrogen at atmospheric pressure and room temperature.

When reduction of the nitro group has been completed (thin-layer chromatography eluted with heptane/AcOEt 1/1), 21 g (0.25 mol) of sodium bicarbonate and 40.2 g (0.236 mol) of benzyl chloroformiate are added at 0° C.

It is shaken for 30 min at 0° C. and filtered over decalite. The decalite is washed with 300 ml of THF and the filtrate liquids are concentrated until the THF has been removed.

200 ml of water is added and 3×200 ml of dichloromethane is extracted. The organic phase is dried and concentrated and the residue is chromatographed on silica gel.

Elution with heptane/AcOEt yields 69.8 g (72%) of the product of the title.

¹H-RMN (CDCl₃, 200 MHz, δ (ppm)): 7.42-7.24 (s.c., 7H); 6.86 (d., 2H); 6.64 (s.a., 1H); 5.18 (s, 2H); 4.60-4.50 (s.c., 4H); 3.10-3.00 (s.c., 4H); 1.46 (s, 9H).

Using the procedure described above the following products are obtained:

### Reference Example No. 9

### 3(R,S)-[(4-benzyloxycarbonylamino-2-fluoro-phenyl)-methyl-amino]-pyrrolidine-1-carboxylic Acid Tert-butyl Ester

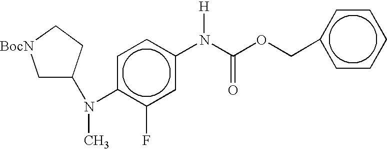

¹H-RMN (CDCl_{3, 200} MHz, δ (ppm)): 7.42-7.26 (s.c. 6H); 7.01-6.92 (s.c., 3H, 2H aromatic+NH); 5.19 (s, 2H);3.86-3.65 (m, 1H); 3.60-3.36 (s.c., 3H); 3.36-3.12 (s.c., 2H); 2.71 (s, 3H); 2.10-1.75 (s.c., 2H); 1.42 (s, 9H).

### Reference Example No. 10

### 3(R,S)-[(4-benzyloxycarbonylamino-2-fluoro-phenyl)-methyl-amino]-azepan-1-carboxylic Acid Tert-butyl Ester

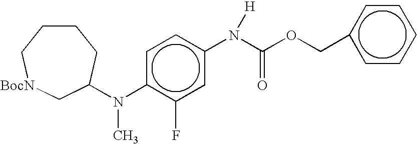

¹H-RMN (CDCl₃, 200 MHz, δ (ppm)): 7.60-7.20 (m, 5H); 7.20-6.80 (m, 3H); 3.95-2.90 (m, 5H); 2.71 (s, 3H); 1.45 and 1.37 (s, 9H); 1.05-2.00 (m, 6H).

### Reference Example No. 11

### 4-[4-(5-(R)-hydroxymethyl-2-oxo-oxazolidin-3-yl)phenyl]-piperazin-1-carboxylic Acid Tert-butyl Ester

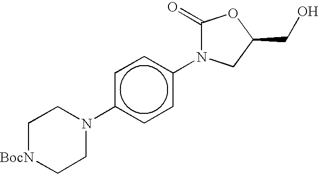

Following a procedure analogous to that of Reference Example No. 4 and using 69.2 g (0.169 mol) of the product obtained in Reference Example No. 8, 44.4 g (70%) of the product of the title is obtained.

¹H-RMN (CDCl₃, 200 MHz, δ (ppm)): 7.42 (d, 2H); 6.92 (d, 2H); 4.80-4.64 (s.c., 1H); 4.02-3.90 (s.c., 3H); 3.80-3.64 (m, 1H); 3.62-3.72 (s.c., 4H); 3.14-3.04 (s.c., 4H); 2.77 (t, 1H, OH); 1.45 (s, 9H).

As in the previous preparation, and following the procedure described in Reference Example No. 4, the following products are obtained:

### Reference Example No. 12

### 3-(R,S)-{[2-fluoro-4-(5-(R)-hydroxymethyl-2oxo-oxazolidin-3-yl)-phenyl]-methyl-amino}-pyrrolidine-1-carboxylic Acid Tert-butyl Ester

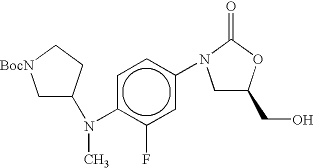

¹H-RMN (CDCl₃, 200 MHz, δ (ppm)): 7.41 (dd, 1H); 7.14-7.00 (s.c., 2H); 4.80-4.64 (m, 1H); 4.02-3.64 (s.c., 5H); 3.62-3.40 (s.c., 2H); 3.38-3.18 (s.c., 2H); 2.78 (s.a., 1H, OH); 2.70 (s, 3H); 2.06-1.80 (s.c., 2H); 1.42 (s, 9H).

### Reference Example No. 13

### 3-(R,S)-{[2-fluoro-4-(5-(R)-hydroxymethyl-2-oxo-oxazolidin-3-yl)-phenyl]-methyl-amino}-azepan-1-carboxylic Acid Tert-butyl Ester

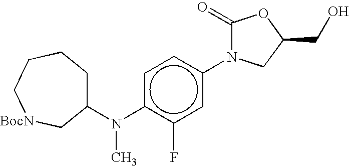

¹H-RMN (CDCl₃, 200 MHz, δ (ppm)): 7.95 (m, 1H); 7.40 (dd, 1H); 7.10 (m, 1H); 4.75 (m, 1H); 4.10-3.00 (m, 9H); 2.73 and 2.76 (s, 3H); 1.39 and 1.46 (s, 9H); 1.20-2.00 (m, 6H).

Following the procedure described in method 1 of Reference Example No. 5 and using respectively the products obtained in reference examples 11 to 13, the following products are obtained:

### Reference Example No. 14

### 4-[4-(5-(R)-azidomethyl-2-oxo-oxazolidin-3-yl)-phenyl]-piperazin-1-carboxylic Acid Tert-butyl Ester

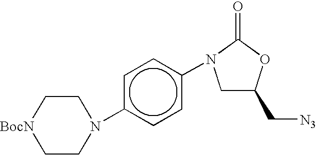

¹H-RMN (DMSO-d₆, 200 MHz, δ (ppm)): 7.44 (d, 2H); 7.02 (d, 2H); 4.96-4.84 (m, 1H); 4.17 (t, 1H); 3.84-3.62 (s.c., 2H); 3.56-3.30 (s.c., 5H); 3.17-3.04 (s.c., 4H); 1.42 (s, 9H).

### Reference Example No. 15

### 3-(R,S)-{[4-(5-(R)-azidomethyl-2-oxo-oxazolidin-3-yl)-2-fluoro-phenyl]-methyl-amino}-pyrrolidine-1-carboxylic Acid Tert-butyl Ester

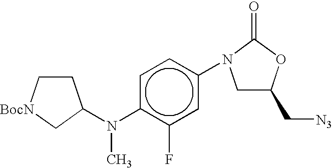

¹H-RMN (CDCl₃, 200 MHz, δ (ppm)): 7.41 (dd, 1H); 7.16-7.01 (s.c., 2H); 4.86-4.72 (m, 1H); 4.06 (t, 1H); 3.95-3.40 (s.c., 6H); 3.38-3.17 (s.c., 2H); 2.73 (s, 3H); 2.10-1.73 (s.c., 2H); 1.45 (s, 9H).

### Reference Example No. 16

### 3-(R,S)-{[4-(5(R)-azidomethyl-2-oxo-oxazolidin-3-yl)-2-fluoro-phenyl]-methyl-amino}-azepan-1-carboxylic Acid Tert-butyl Ester

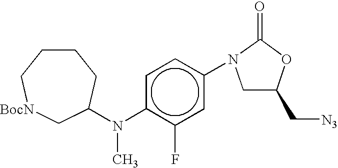

¹H-RMN (CDCl₃, 200 MHz, δ (ppm)): 7.35 (m, 1H); 7.20-6.80 (m, 2H); 4.75 (m, 1H); 4.05 (t, 1H); 3.95-3.00 (m, 8H); 2.74 (m, 3H); 2.00-1.00 (m, 6H); 1.46 and 1.39 (s, 9H).

### Reference Example No. 17

### 4-[2-Fluoro-4-(5-(R)-{[isoxazol-3-yl-(2,2,2-trichloro-ethoxycarbonyl)-amino]-methyl}-2-oxo-oxazolidin-3-yl)-phenyl]-piperazin-1-carboxylic Acid Tert-butyl Ester

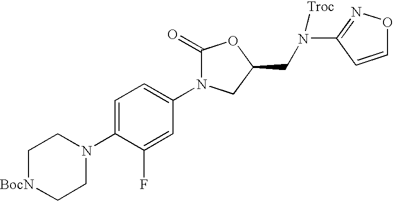

3.4 g (13 mmol) of 3-(2,2,2-trichloroethoxycarbonylamino)-isoxazol (prepared accordingto WO 0021960) is dissolved in 100 ml of DMF, and 536 mg (14.3 mmol) of sodium hydride (60% paste) is added in portions and stirred for 30 minutes. 6 g (12.7 mmol) of 4-{2-Fluoro-4-[2-oxo-5-(R)-(toluene-4-sulphonylxymethyl)-oxazolidin-3-yl]-phenyl}-piperazine-1-carboxylic acid tert-butyl ester (obtained according to U.S. Pat. No. 5,547,950) is then added dissolved in 30 ml of DMF.

The reaction is heated to 90° C. for 20 h. It is allowed to cool and is poured onto 500 ml of water. It is extracted with 3×250 ml of a 4/1 mixture of toluene/ethyl acetate. The organic phase is dried and concentrated and the residue is chromatographed on silica gel.

Elution with Heptane/Ethyl acetate 7/3 yields 2.5 g of the product of the title.

¹H-RMN (CDCl₃, 200 MHz, δ (ppm)): 8.34 (d, 1H) 7.45 (dd, 1H); 7.12 (m, 1H); 6.95 (m, 2H); 5.15 (m, 1H); 4.90 (m, 2H); 4.50 (dd, 1H); 4.25 (dd, 1H); 4.13 (t, 1H); 3.85 (dd, 1H); 3.60 (m, 4H); 3.00 (m, 4H); 1.49 (s, 9H).

### Reference Example No. 18

### 3-(3-Fluoro-4-piperazin-1-yl-phenyl)-5-(R)-hydroxymethyl-oxazolidin-2-one

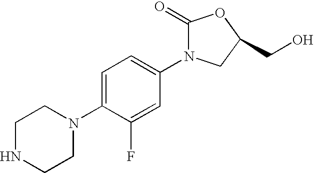

To 5 g (0.0126 mol) of 4-[2-fluoro-4-(5-(R)-hydroxymethyl-2-oxo-oxazolidin-3-yl)-phenyl]-piperazin-1-carboxylic acid tert-butyl ester (obtained according to U.S. Pat. No. 5,547,950) in 100 ml of ethanol is added 2.6 g (0.0139 mol) of para-toluenesulphonic acid and this is heated to reflux for 16 h. It is concentrated to dryness and the residue is chromatographed on silica gel (80 g) to the upper part of which alumina (20 g) is added.

Elution with dichloromethane/ethanol/ammonium hydroxide (90/10/1%) yields 1.6 g of the product of the title.

¹H-RMN (CDCl₃, 200 MHz, δ (ppm)): 7.50 (d.d., 1H); 7.24-7.00 (s.c, 2H); 4.70 (m, 1H); 4.04 (t, 1H); 3.82-3.42 (s.c, 3H); 2.86 (s.a, 8H).

### Reference Example No. 19

### 5-(R)-azidomethyl-3-(3-fluoro-4-piperazin-1-yl-phenyl)-oxazolidin-2-one

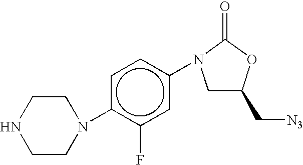

To 5 g (0.011 mol) of 4-[4-(5-(R)-azidomethyl-2-oxo-oxazolidin-3-yl)-phenyl]-piperazin-1-carboxylic acid tert-butyl ester (obtained according to U.S. Pat. No. 5,547,950) in 100 ml of ethanol is added 2.4 g (0.013 mol) of p-toluenesulphonic acid.

It is heated to reflux for 16 h. Once the reaction has ended it is concentrated to dryness and the residues pass through a column of silica gel (100 g) containing 25 g of alumina in the upper part.

Elution with dichloromethane/ethanol/ammonium hydroxide (80/20/1%) yields 3.5 g of the product of the title.

¹H-RMN (CDCl₃, 200 MHz, δ (ppm)): 7.42 (dd, 1H); 7.10 (dd, 1H); 6.94 (t, 1H); 4.84-4.76 (m, 1H); 4.05 (t, 1H); 3.83-3.50 (s.c, 3H); 3.03 (s, 3H).

### Reference Example No. 20

### 4-{4-5-(S)-(acetylamino-methyl)-2-oxo-oxazolidin-3-yl]-phenyl}-piperazin-1-carboxylic Acid Tert-butyl Ester

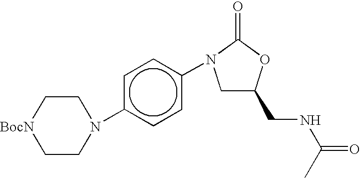

To 40 g (0.0668 mol) of the product of Reference Example No. 14 in 1,000 ml of ethyl acetate is added 4 g of 10% Pd/C paste and it is placed under atmosphere of hydrogen at atmospheric pressure and room temperature. When reduction of the azide group has finished (thin-layer chromatography), it is cooled to 0° C. and 8.4 ml (0.103 mol) of pyridine and 13.4 ml (0.103 mol) of acetic anhydride are added.

It is stirred at 0° C. for 30 min and then for 16 h at room temperature. It is filtered over decalite and the filtration liquids are concentrated to dryness.

The residue is chromatographed on silica gel. Elution with dichloromethane/ethanol 95/5 yields 27 g (97%) of the product of the title.

¹H-RMN (DMSO, 200 MHz, δ (ppm)): 8.30 (t, 1H, NH); 7.41 (d, 2H); 7.00 (d, 2H); 4.80-4.60 (m, 1H); 4.10 (t, 1H); 3.72 (t, 1H); 3.55-3.38 (s.c., 6H); 3.15-3.03 (s.c., 4H); 1.83 (s, 3H); 1.42 (s, 9H).

Following the procedure described above and using the products of reference examples No. 15 and No. 16, the following products are obtained:

### Reference Example No. 21

### 3-(R,S)-({4-5-(S)-(acetylamino-methyl)-2-oxo-oxazolidin-3-yl]-2-fluoro-phenyl}-methyl-amino)-pyrrolidine-1-carboxylic Acid Tert-butyl Ester

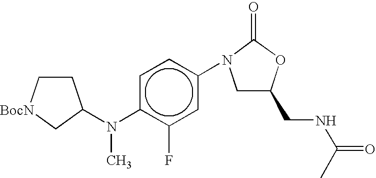

¹H-RMN (CDCl₃, 200 MHz, δ (ppm)): 7.41 (dd, 1H); 7.10-7.00 (s.c., 2H); 6.61 (t, 1H, NH); 4.82-4.70 (m, 1H); 4.02 (t, 1H); 3.97-3.40 (s.c., 6H); 3.40-3.18 (s.c., 2H); 2.75 (s, 3H); 2.10-1.80 (s.c., 2H); 1.42 (s, 9H).

### Reference Example No. 22

### 3-(R,S)-({4-[5-(S)-(acetylamino-methyl)-2-oxo-oxazolidin-3-yl]-2-fluoro-phenyl}-methyl-amino]-azepan-1-carboxylic Acid Tert-butyl Ester

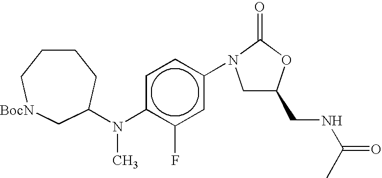

¹H-RMN (CDCl₃, 200 MHz, δ (ppm)): 7.35 (dd, 1H); 7.15-6.85 (m, 2H); 6.45 (m, 1H); 4.75 (m, 1H); 4.01 (t, 1H); 3.90-3.00 (m, 8H); 2.76 and 2.23 (s, 3H); 2.03 (s, 3H); 1.46 and 1.39 (s, 9H); 2.00-1.10 (m, 6H).

### Reference Example No. 23

### 4-[4-(5-(S)-aminomethyl-2-oxo-oxazolidin-3-yl)-2-fluoro-phenyl]-piperazin-1-carboxylic Tert-butyl Ester

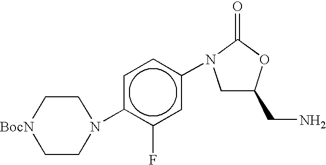

To 30 g (0.071 mol) of 4-[4-(5-(R)-azidomethyl-2-oxo-oxazolidin-3-yl)-phenyl]-piperazin-1-carboxylic acid tert-butyl ester (obtained according to U.S. Pat. No. 5,547,950) in 300 ml of ethanol is added 3 g of 10% Pd/C paste and it is placed under atmosphere of hydrogen at atmospheric pressure and room temperature. When the reaction has finished (thin-layer chromatography eluted with dichloromethane-ethanol 95/5) it is filtered over decalite and the decalite washed with 50 ml of ethanol.

The filtering liquids are concentrated to dryness and the residue is chromatographed on silica gel.

Elution with dichloromethane/ethanol/ammonium hydroxide 90/10/1% yields 14 g (50%) of the product of the title.

¹H-RMN (CDCl₃, 200 MHz, δ (ppm)): 7.47 (dd, 1H); 7.13 (dd, 1H); 6.94 (t, 1H); 4.75-4.60 (m, 1H); 4.01 (t, 1H); 3.82 (dd, 1H); 3.62-3.51 (s.c., 4H); 3.20-2.90 (s.c., 6H); 1.50 (s, 9H); 1.40 (s.a., 2H, NH₂).

### Reference Example No. 24

### 4-{2-fluoro-4-[5-(R)-(1-(R,S)-hydroxy-prop-2-inyl)-2-oxo-oxazolidin-3-yl]-phenyl}-piperazin-1-carboxylic Acid Tert-butyl Ester

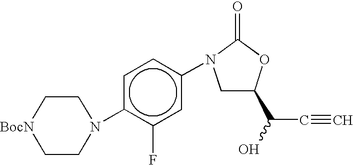

To 2.4 g (32.2 mmol) of tert-butanol in 30 ml of dry tetrahydrofuran, cooled to −10° C., is added 9.2 ml (23 mmol) of n-Buli (2.5 M in hexane).

It is stirred for 30 min and allowed to reach a temperature of 0° C. 4.49 g (10 mmol) of 4-(4-benzyloxycarbonylamino-2-fluoro-phenyl)-piperazin-1-carboxylic acid tert-butyl ester (obtained according to U.S. Pat. No. 5,547,950) is then added, dissolved in 10 ml of dry dimethylformamide.

After stirring for 10 min at 0° C., 3.4 g (12.5 mmol) of 2,3-hydroxy-pent-4-inyl p-toluenesulphonate (obtained according to EP 1029854 A1) dissolved in 5 ml of DMF is then added dropwise.

It is allowed to reach room temperature and stirred for 16 h. It is poured onto 200 ml of saturated solution of sodium bicarbonate and extracted with 3×150 ml of ethyl acetate. The organic extracts are washed with 150 ml of water. The organic phase is dried and concentrated and the residue is chromatographed on silica gel.

Elution with ethyl acetate/heptane 1/1 yields 2.6 g (62%) of the product of the title.

¹H-RMN (CDCl₃, 200 MHz, δ (ppm)): 7.45 (dd, 1H); 7.15 (m, 1H); 6.95 (t, 1H); 4.75 (m, 2H); 4.30-2.90 (m, 3H); 3.60 (m, 4H); 3.00 (m, 4H); 2.53 (d, 1H); 1.48 (s, 9H).

Following the procedure described in reference examples 18 and 19 and using respectively the compounds obtained in reference examples 17 and 20 to 24 the following products are obtained:

### Reference Example No. 25

### [3-(3-Fluoro-4-piperazin-1-yl-phenyl)-2-oxo-oxazolidin-5-ylmethyl]-isoxazol-3-yl-carbamate of 2,2,2-trichloro-ethyl

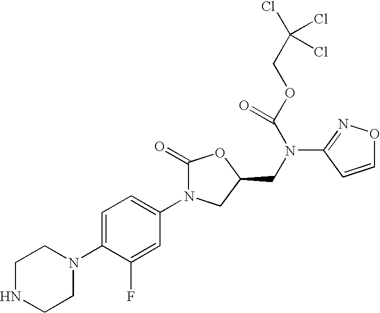

¹H-RMN (CDCl₃, 200 MHz, δ (ppm)): 8.34 (d, 1H); 7.42 (dd, 1H); 7.10 (dd, 1H); 6.95 (m, 2H); 5.15 (m, 1H); 4.95 (m, 2H); 4.52 (dd, 1H); 4.25 (dd, 1H); 4.12 (t, 1H); 3.80 (dd, 1H); 3.12 (m, 8H).

### Reference Example No. 26

### N-[2-oxo-3-(4-piperazin-1-yl-phenyl)-oxazolidin-5-(S)-ylmethyl]acetamide

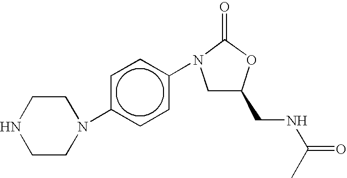

¹H-RMN (DMSO-d₆, 200 MHz, δ (ppm)): 8.30 (t, 1H, NH); 7.41 (dd, 2H); 7.00 (dd, 2H); 4.80-4.60 (m, 1H); 4.06 (t, 1H); 3.71 (dd, 1H); 3.42 (t, 2H); 3.30-3.10 (s.c., 8H); 1.82 (s, 3H).

### Reference Example No. 27

### N-{3(R,S)-[3-fluoro-4-(methyl-pyrrolidine-3-yl-amino)-phenyl]-2-oxo-oxazolidin-5-(S)-ylmethyl}-acetamide

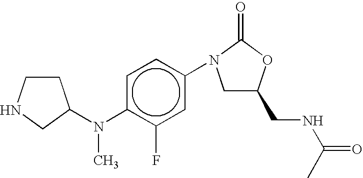

¹H-RMN (CDCl₃, 200 MHz, δ (ppm)): 7.39 (dd, 1H); 7.10-6.97 (s.c., 2H); 6.49 (t, 1H, NH); 4.83-4.70 (m , 1H); 4.02 (t, 1H); 3.90-3.60 (s.c., 4H); 3.13-2.80 (s.c., 4H); 2.72 (s, 3H); 2.02 (s, 3H); 2.00-1.65 (s.c., 2H).

### Reference Example No. 28

### N-{3(R,S)-[4-(azepan-3-yl-methyl-amino)-3-fluoro-phenyl]-2-oxo-oxazolidin-5-(S)-ylmethyl}-acetamide

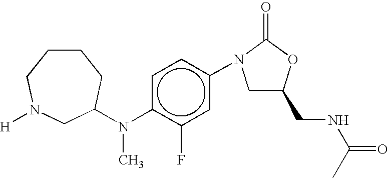

¹H-RMN (CDCl₃, 200 MHz, δ (ppm)): 7.35 (dd, 1H); 7.05 (m, 1H); 6.90 (t, 1H); 6.75 (t, 1H, NH); 4.75 (m, 1H); 4.00 (t, 1H); 3.90-3.30 (m, 4H); 3.20-2.60 (m, 4H); 2.72 (s, 3H); 2.30 (s.a., 1H); 2.02 (s, 3H); 1.90-1.00 (m, 6H).

### Reference Example No. 29

### p-toluenesulphate of 5-(S)-aminomethyl-3-(3-fluoro-4-piperazin-1-yl-phenyl)-oxazolidin-2-one

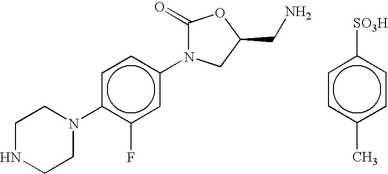

¹H-RMN (DMSO-d₆, 200 MHz, δ (ppm)): 7.56 (dd, 1H); 7.50 (d, 2H); 7.22-7.06 (s.c., 4H); 4.90-4.74 (m, 1H); 4.14 (t, 1H); 3.84-3.76 (m, 1H); 3.25-3.05 (s.c., 10H); 2.26 (s, 3H).

### Reference Example No. 30

### 3-(3-fluoro-4-piperazin-1-yl-phenyl)-5-(R)-(1-(R,S)-hydroxy-prop-2-inyl)-oxazolidin-2-one

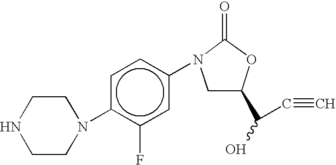

¹H-RMN (DMSO-d₆, 200 MHz, δ (ppm)): 7.50 (m, 1H); 7.20 (m, 1H); 7.03 (m, 1H); 6.15 (s.a., 1H); 4.70 (m, 1H); 4.52 (m, 1H); 4.10 (t, 1H); 3.85 (m, 1H); 3.25 (m, 1H); 3.23 (s.a., 1H).

### Reference Example No. 31

### 7-(4-{-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydroquinoline-3-carboxylic Acid Diacethoxyboron Chelate

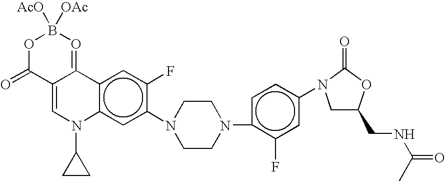

To 1 g (3 mmol) of N-[3-(3-Fluoro-4-piperazin-1-yl-phenyl)-2-oxo-oxazolidin-5-(S)-ylmethyl]-acetamide (obtained according to U.S. Pat. No. 5,547,950) in 30 ml of acetonitrile are added 1.22 g of 7-chloro-1-cyclopropyl-6-fluoro-4-oxo-1,4-dihydro-quinoline-3-carboxylic acid diacethoxyboron chelate (obtained according to WO 8807998) and 0.43 ml (3 mmol) of triethylamine.

The reaction is heated to reflux for 16 h. It is concentrated to dryness and the residue is chromatographed on silica gel.

Elution with dichloromethane/ethanol 90/10 yields 0.8 g of the product of the title.

¹H-RMN (CDCl₃, 200 MHz, δ (ppm)): 9.04 (s, 1H); 8.10 (d, 1H); 7.56-7.44 (s.c., 2H); 7.08 (dd, 1H); 6.97 (t, 1H); 6.38 (t, 1H, NH); 4.82-4.68 (m, 1H); 4.01 (t, 1H); 3.90-3.56 (s.c., 8H); 3.30-3.20 (s.a., 4H); 2.04 (s, 6H); 2.02 (s, 3H); 1.90-1.20 (s.c., 2H).

### Reference Example No. 32

### 7-[3-(R,S)-({4-[5-(S)-(acetylamino-methyl)-2-oxo-oxazolidin-3-yl]-2-fluoro-phenyl}-methylamino)-azepan-1-yl]-1-cyclopropyl-6-fluoro-4-oxo-1,4-dihydro-quinoline-3-carboxylic Acid Diacethoxyboron Chelate

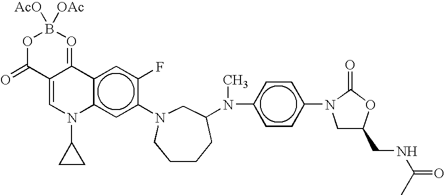

Following the procedure of the previous example and using the product obtained in Reference Example No. 28, the product of the title is obtained.

¹H-RMN (DSMO-d₆, 200 MHz, δ (ppm)): 8.94 (s, 1H); 8.30 (t, 1H); 7.90 (d, 1H); 7.60-7.40 (m, 2H); 7.30-7.10 (m, 2H); 4.75 (m, 1H); 4.30-3.40 (m, 10H); 2.80 (s, 3H); 2.10-1.05 (m, 10H); 1.93 (s, 6H); 1.88 (s, 3H).

### Reference Example No. 33

### 7-{4-[4-(5-(S)-aminomethyl-2-oxo-oxazolidin-3-yl)-2-fluoro-phenyl]-piperazin-1-yl}-1-cyclopropyl-6-fluoro-4-oxo-1,4-dihydro-quinoline-3-carboxylic Acid Diacethoxyboron Chelate

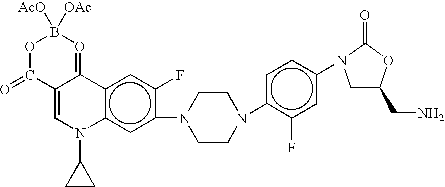

Following the procedure described in Reference Example No. 31 and using the product obtained in Reference Example No. 29 and using 2 equivalents of triethylamine instead of only one equivalent, the product of the title is obtained.

¹H-RMN (DSMO-d₆, 200 MHz, δ (ppm)): 9.03 (s, 1H) 8.04 (d, 1H); 7.82 (d, 1H); 7.59 (dd, 1H); 7.24 (dd, 1H); 7.17 (t, 1H); 4.70-4.56 (m, 1H); 4.14 (s.a., 1H); 4.08 (t, 1H); 3.84 (dd, 1H); 3.64 (s.a., 4H); 3.23 (s.a., 4H); 2.90-2.70 (s.c., 2H); 2.20 (s.a., 2H, NH₂); 1.90 (s, 6H); 1.50-1.20 (s.c., 4H).

### Reference Example No. 34

### 7-(4-{5-[5-(S)-(acetylamino-methyl)-2-oxo-oxazolidin-3-yl]-2-fluoro-phenyl}-piperazin-1-yl)-1-ethyl-6,8-difluoro-4-oxo-1,4-dihydro-quinoline-3-carboxylic Acid Boron Difluoride Chelate

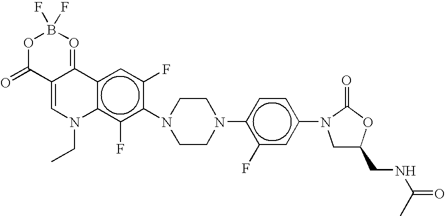

Following a procedure analogous to that described in Reference Example No. 31 and using 1-ethyl-6,7,8-trifluoro-4-oxo-1,4-dihydro-quinoline-3-carboxylic acid boron difluoride chelate (obtained according to WO 8807998) the product of the title is obtained.

¹H-RMN (DSMO-d₆, 200 MHz, δ (ppm)): 9.44 (s, 1H); 8.27 (t, 1H, NH); 8.09 (d, 1H); 7.54 (dd, 1H); 7.30-7.06 (s.c., 2H); 5.00-4.60 (s.c., 3H); 4.10 (t, 1H); 3.80-2.95 (s.c., 11H); 1.85 (s, 3H); 1.55 (t, 3H).

### Reference Example No. 35

### 7-(4-{4-[5-(S)-(acetylamino-methyl)-2-oxo-oxazolidin-3-yl]-2-fluoro-phenyl}-piperazin-1-yl)-1-ethyl-6-fluoro-4-oxo-1,4-dihydro-quinoline-3-carboxylic Acid Boron Difluoride Chelate

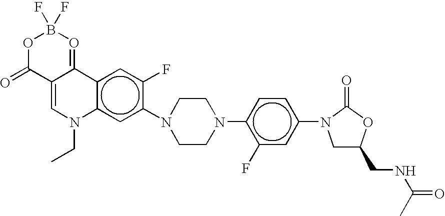

Following a procedure analogous to that described in Reference Example No. 31 and using 7-chloro-1-ethyl-6-fluoro-4-oxo-1,4-dihydro-quinoline-3-carboxylic acid boron difluoride chelate (obtained according to JP 59122470) the product of the title is obtained.

¹H-RMN (DSMO-d₆, 200 MHz, δ (ppm)): 9.42 (s, 1H); 8.30 (t, 1H, NH); 8.17 (d, 1H); 7.60-7.40 (s.c., 2H); 7.25-7.05 (s.c., 2H); 4.90 (c, 2H); 4.80-4.60 (m, 1H); 4.14 (t, 1H); 3.80-2.90 (s.c., 11H); 1.84 (s, 3H); 1.52 (t, 3H).

### Reference Example No. 36

### 9-(4-{4-[5-(S)-(acetylamino-methyl)-2-oxo-oxazolidin-3-yl]-phenyl}-piperazin-1-yl)-8-fluoro-3-methyl-6-oxo-2,3-dihydro-6H-1-oxa-3-aza-phenalen-5-carboxylic Acid Boron Difluoride Chelate

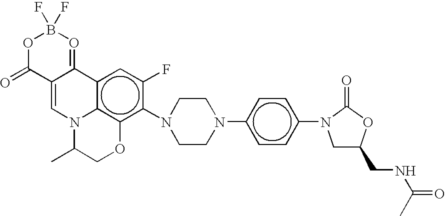

Using 8,9-difluoro-3-methyl-6-oxo-2,3-dihydro-6H-1-oxa-3a-aza-phenalen-5-carboxylic acid boron difluoride chelate (obtained according to JP 58029789) and following a procedure analogous to that described in Reference Example No. 31 the product of the title is obtained.

¹H-RMN (DSMO-d₆, 200 MHz, δ (ppm)): 9.44 (s, 1H); 8.30 (t, 1H, NH); 7.84 (d, 1H); 7.43 (d, 2H); 7.05 (d, 2H); 5.30-5.10 (m, 1H); 4.80-4.30 (s.c., 3H); 4.10 (t, 1H); 3.80-3.15 (s.c., 11H); 1.84 (s, 3H); 1.58 (d, 3H).

### Reference Example No. 37

### 9-[3-({4-[5-(S)-(acetylamino-methyl)-2-oxo-oxazolidin-3-yl]-2-fluorophenyl}-methyl-amino)-pyrrolidone-1-yl]-8-fluoro-3-methyl-6-oxo-2,3-dihydro-6H-1-oxa-3a-aza-phenalen-5-carboxylic Acid Boron Difluoride Chelate

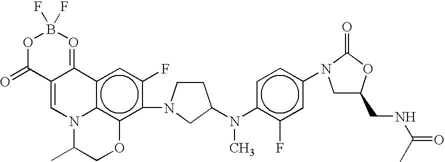

In a manner analogous to the previous example and using the compound obtained in Reference Example No. 27 the product of the title is obtained.

¹H-RMN (DSMO-d₆, 200 MHz, δ (ppm)): 9.36 (s, 1H); 8.25 (t, 1H, NH); 7.74 (d, 1H); 7.50 (dd, 1H); 7.30-7.10 (s.c., 2H); 5.20-3.00 (s.c., 13H); 2.78 (s, 3H); 1.82 (s, 3H); 2.20-1.80 (s.c., 2H); 1.50 (d, 3H).

### Reference Example No. 38

### 4-{4-[4(5-(S)-aminomethyl-2-oxo-oxazolidin-3-yl)-2-fluoro-phenyl]-piperazin-1-yl}-1-cyclopropyl-6-fluoro-4-oxo-1,4-dihydro-quinoline-3-carboxylic Acid

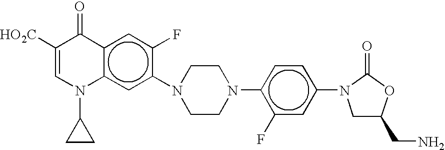

Method 1:

To 13.3 g (0.02 mol) of the product obtained in Reference Example No. 33 in 300 ml of acetonitrile and 300 ml of water is added 96 ml (0.096 mol) of sodium hydroxide 1N.

It is stirred at room temperature for 2 h. The acetonitrile is concentrated in a rotovapor and to the resulting aqueous solution is added 96 ml of hydrochloric acid 1 N.

The precipitate formed is filtered to yield 2.8 g. The filtering liquids are extracted with 4×200 ml of dichloromethane/ethanol 90/10. The extracts are dried and and concentrated, thus yielding a further 6.8 g of the product of the title.

¹H-RMN (DSMO-d₆, 200 MHz, δ (ppm)): 8.70 (s, 1H); 7.95 (d, 1H); 7.63 (d, 1H); 7.58 (dd, 1H); 7.26-7.10 (s.c., 2H); 4.80-4.60 (m, 1H); 4.08 (t, 1H); 3.96-3.80 (s.c., 2H); 3.50 (s.a., 4H+NH₂); 3.23 (s.a., 4H); 3.00-2.80 (s.c., 2H); 1.42-1.15 (s.c., 4H).

Method 2:

To 40 mg of the product obtained by method 1 of Reference Example No. 5, dissolved in 10 ml of ethanol, is added 0.10 mg of 10% Pd/C paste, and it is placed under atmosphere of hydrogen at atmospheric pressure and room temperature. When the reaction finishes it is filtered over decalite, which is washed with 2×10 ml of ethanol.

The filtering liquids are concentrated to dryness and thus yield 20 mg of a product identical to that obtained by method 1.

### Compounds of General Formula (I)

### Example 1

### 7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic Acid

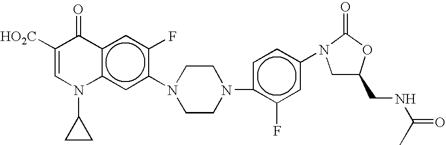

To 0.8 g (1.13 mol) of the product of Reference Example No. 31 in 20 ml of water and 20 ml of acetonitrile is added 5.6 ml of sodium hydroxyde 1N, and it is stirred at room temperature for 1 h.

The acetonitrile is concentrated and the aqueous phase is acidified with 5.6 ml of hydrochloric acid 1N.

It is extracted with 3×50 ml of dichloromethane/ethanol 9/1.

The organic phase is dried and concentrated. The residue is stirred for 10 min with 2-propanol and the precipitated solid is filtered. Thus are obtained 290 mg of the product of the title.

¹H-RMN (DSMO-d₆, 200 MHz, δ (ppm)): 8.72 (s, 1H); 8.33 (t, 1H, NH); 7.99 (d, 1H); 7.64 (d, 1H); 7.58 (dd, 1H); 7.30-7.10 (s.c., 2H); 4.84-4.64 (m, 1H); 4.16 (t, 1H); 3.90-2.90 (s.c., 12H); 1.90 (s, 3H); 1.44-1.16 (s.c., 4H).

### Example 2

### 7-[3-({4-[5-(S)-(acetylamino-methyl)-2-oxo-oxazolidin-3-yl]-2-fluoro-phenyl}-methyl-amino)-azepan-1-yl]-1-cyclopropyl-6-fluoro-4-oxo-1,4-dihydro-quinoline-3-carboxylic Acid

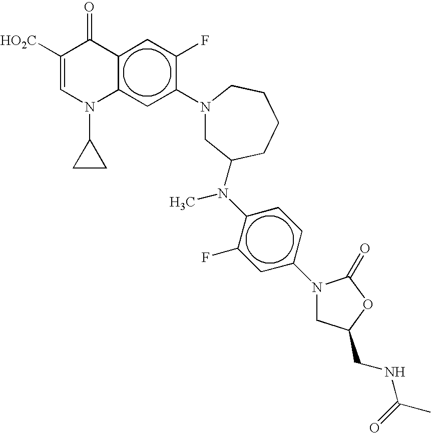

It is obtained by following the procedure of Example 1 and using the product obtained in Reference Example No. 32.

¹H-RMN (DSMO-d₆, 200 MHz, δ (ppm)): 8.59 (s, 1H); 8.30 (t, 1H, NH); 7.80 (d, 1H); 7.50 (dd, 1H); 7.30 (d, 1H); 7.25-7.05 (s.c., 2H); 4.75 (m, 1H); 4.20-3.20 (m, 10H); 2.76 (s, 3H); 2.20-1.00 (m, 10H); 1.86 (s, 3H).

### Example 3

### 7-(4-{4-[5-(S)-(acetylamino-methyl)-2-oxo-oxazolidin-3-yl]-2-fluoro-phenyl}-piperazin-1-yl)-1-ethyl-6,8-difluoro-4-oxo-1,4-dihydro-quinoline-3-carboxylic Acid

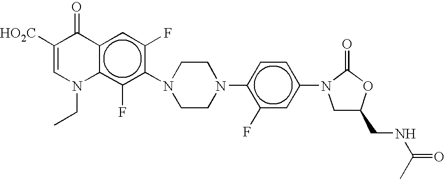

To 1.9 g (3mmol) of the product obtained in Reference Example No. 34 in 100 ml of ethanol and 2.5 ml of water is added 10 ml of triethylamine, and it is heated to reflux for 16 h.

The precipitated salts are filtered. The filtering liquids are concentrated to dryness and the residue is treated with 50 ml of water and the pH adjusted to 5 by addition of hydrochloric acid 1N.

It is extracted with 3×75 ml of dichloromethane/ethanol 9/1. The organic phase is dried and concentrated. Thus are obtained 1.2 g of a white solid.

¹H-RMN (DSMO-d₆, 200 MHz, δ (ppm)) 8.94 (s, 1H) 8.30 (t, 1H, NH); 7.87 (d, 1H); 7.50 (dd, 1H); 7.25-7.02 (s.c., 2H); 4.80-4.30 (s.c., 3H); 4.10 (t, 1H); 3.80-3.20 (s.c., 7H); 3.10 (s.a., 4H); 1.82 (s, 3H); 1.42 (t, 3H).

### Example 4

### 7-(4-{4-[5-(S)-(acetylamino-methyl)-2-oxo-oxazolidin-3-yl]-2-fluoro-phenyl}-piperazin-1-yl)-1-ethyl-6-fluoro-4-oxo-1,4-dihydro-quinoline-3-carboxylic Acid

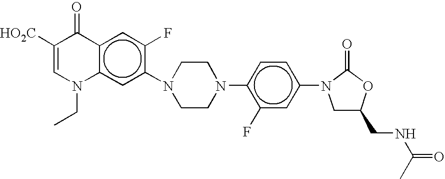

Following the procedure of the previous example and using the product obtained in Reference Example No. 35 the product of the title is achieved.

¹H-RMN (DSMO-d₆, 200 MHz, δ (ppm)): 8.99 (s, 1H) 8.30 (t, 1H, NH); 7.96 (d, 1H); 7.54 (d, 1H); 7.20-7.05 (s.c., 3H); 5.00-4.56 (s.c., 3H); 4.14 (t, 1H); 3.90-3.10 (s.c., 11H); 1.82 (s, 3H); 1.60-1.35 (s.a., 3H).

### Example 5

### 9-(4-{4-[5-(S)-(acetylamino-methyl)-2-oxo-oxazolidin-3-yl]-phenyl}-piperazin-1-yl)-8-fluoro-3-methyl-6-oxo-2,3-dihydro-6H-1-oxa-3a-aza-phenalen-5-carboxylic Acid

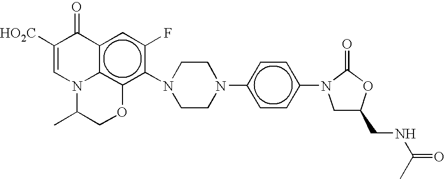

Following the procedure described in Example 3 and using the product obtained in Reference Example No. 36 the product of the title is achieved.

¹H-RMN (DSMO-d₆, 200 MHz, δ (ppm)): 9.00 (s, 1H); 8.26 (t, 1H, NH); 7.62 (d, 1H); 7.41 (d, 2H); 7.02 (d, 2H); 5.05-4.90 (m, 1H); 4.80-4.75 (s.c., 2H); 4.41 (d, 1H); 4.10 (t, 1H); 3.80-3.00 (s.c., 11H); 1.84 (s, 3H); 1.46 (d, 3H).

### Example 6

### 9-[3-({4-[5-(S)-(acetylamino-methyl)-2-oxo-oxazolidin-3-yl]-2-fluoro-phenyl}-methyl-amino)-pyrrolidin-1-yl]-8-fluoro-3-methyl-6-oxo-2,3-dihydro-6H-1-oxa-3a-aza-phenalen-5-carboxylic Acid

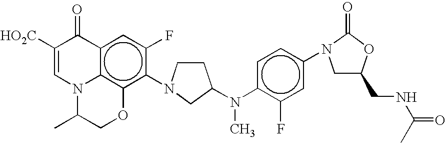

Following the procedure of Example No. 3 and using the product of Reference Example No. 37 the product of the title is obtained.

¹H-RMN (DSMO-d₆, 200 MHz, δ (ppm)): 8.92 (s, 1H); 8.30 (t, 1H, NH); 7.60-7.40 (s.c., 2H); 7.30-7.10 (s.c., 2H); 4.95-4.80 (m, 1H); 4.80-4.45 (s.c., 3H); 4.40-4.20 (s.c., 1H); 4.10 (t, 1H), 4.02-3.20 (s.c., 7H); 2.70 (s, 3H); 2.20-1.90 (s.c., 2H); 1.84 (s, 3H); 1.45 (s.a., 3H).

### Example 7

### 9-(4-{4-[5-(S)-(acetylamino-methyl)-2-oxo-oxazolidin-3-yl]-2-fluoro-phenyl}-piperazin-1-yl)-8-fluoro-3-methyl-6-oxo-2,3-dihydro-6H-1-oxa-3a-aza-phenalene-5-carboxylic Acid

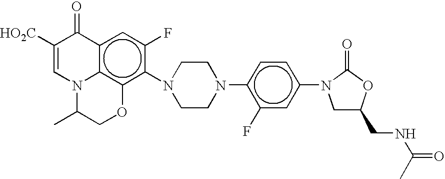

To 1.6 g (5mmol) of 8,9-difluoro-3-methyl-6-oxo-2,3-dihydro-6H-1-oxa-3a-aza-phenalene-5-carboxylic acid boron difluoride chelate and 1.7 g (5 mmol) of N-[3-(3-Fluoro-4-piperazin-1-yl-phenyl)-2-oxo-oxazolidin-5-(S)-ylmethyl]-acetamide (obtained according to U.S. Pat. No. 5,547,950) in 50 ml of N-methyl-pyrrolidin-2-one is added 0.7 ml (5 mmol) of triethylamine and it is heated at 110° C. for 16 h.

The solvent is distilled under vacuum and the residue is stirred for 30 min with dichloromethane/ethanol, precipitating a solid which is filtered and yields 1.2 g (40%) of pure product.

¹H-RMN (DSMO-d₆, 200 MHz; δ (ppm)): 9.00 (s, 1H); 8.25 (t, 1H, NH); 7.62 (d, 1H); 7.52 (dd, 1H); 7.30-7.10 (s.c., 2H); 4.99 (m, 1H); 4.80-4.60 (m, 1H); 4.62 (d, 1H); 4.40 (d, 1H); 4.10 (t, 1H); 3.80-3.60 (m, 1H); 3.60-2.80 (s.c., 10H); 1.84 (s, 3H); 1.50 (d, 3H).

### Example 8

### 7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic Acid

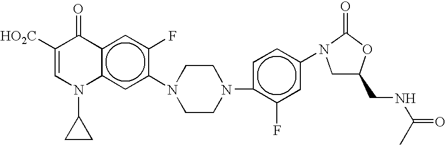

To 6 g (0.011 mol) of the product of Reference Example No. 38 in 100 ml of pyridine is added 2.8 ml (0.022 mol) of acetic anhydride. It is heated at 50° C. for 2 h. The pyridine is concentrated to dryness and to the residue is added 200 ml of water and it is stirred for 5 min. The precipitated solid is filtered and dissolved in dichloromethane and chromatographed on silica gel. Elution with dichloromethane-ethanol 90/10 yields 4 g (63%) of pure product identical to that obtained in Example 1.

### Example 9

### 1-cyclopropyl-6-fluoro-7-[4-(2-fluoro-4-{5-(S)-[(3-methyl-thioureido)-methyl]-2-oxo-oxazolidin-3-yl}-phenyl)-piperazin-1-yl]-4-oxo-1,4-dihydro-quinoline-3-carboxylic Acid

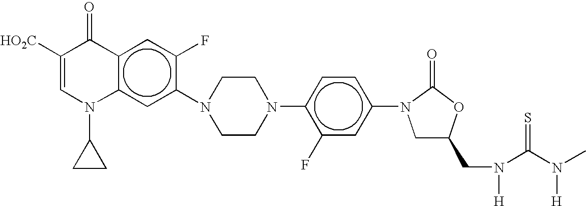

To 0.81 g (1.5 mmol) of the product of Reference Example No. 38 in 10 ml of pyridine is added 0.22 g (3 mmol) of methylisothiocyanate. It is heated at 60° C. for 10 minutes. It is concentrated to dryness and the residue is stirred for 20 min with 30 ml of water. The precipitated solid is filtered and 0.5 g of pure product is obtained.

¹H-RMN (DSMO-d₆, 200 MHz, δ (ppm)): 8,70 (s, 1H); 7.98 (d, 1H); 7.82 (t, 1H, NH); 7.80-7.50 (s.a., 1H, NH); 7.64 (d, 1H); 7.56 (dd, 1H); 7.30-7.10 (s.c., 2H); 4.95-4.80 (m, 1H); 4.16 (t, 1H); 4.00-3.70 (s.a., 4H); 3.60-3.40 (s.a., 4H); 3.30-3.10 (s.a., 4H); 2.82 (s.a., 3H); 1.44-1.16 (s.c., 4H).

### Example 10

### 1-cyclopropyl-7-[4-(4-{5-(S)-[(3-ethyl-ureido)-methyl]-2-oxo-oxazolidin-3-yl}-2-fluoro-phenyl)-piperazin-1-yl]-6-fluoro-4-oxo-1,4-dihydro-quinoline-3-carboxylic Acid

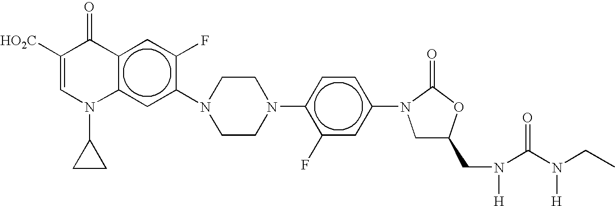

In a similar way to the previous Example and replacing the methylisothiocyanate by ethylisocyanate the product of the title is obtained.

¹H-RMN (DSMO-d₆, 200 MHz, δ (ppm)): 8.70 (s, 1H); 7.96 (d, 1H); 7.66 (d, 1H); 7.58 (dd, 1H); 7.30-7.10 (s.c., 1H); 6.22 (t, 1H, NH); 5.99 (t, 1H, NH); 4.80-4.64 (s.c., 1H); 4.10 (t, 1H); 3.90-3.78 (m, 1H); 3.72 (dd, 1H); 3.60-3.20 (s.c., 10H); 3.10-2.90 (s.c., 2H); 1.44-1.10 (s.c., 4H); 0.98 (t, 3H).

### Example 11

### 1-cyclopropyl-7-(4-{4-[5-(S)-(ethoxycarbonylamino-methyl)-2-oxo-oxazolidin-3-yl]-2-fluoro-phenyl}-piperazin-1-yl)-6-fluoro-4-oxo-1,4-dihydro-quinoline-3-carboxylic Acid

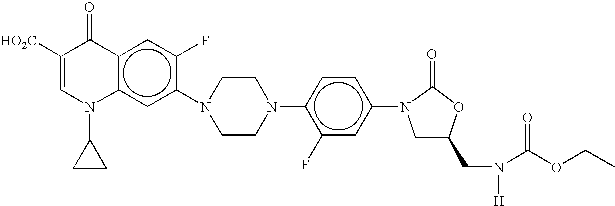

To 0.81 g of the product of Reference Example No. 38 in 20 ml of tetrahydrofuran are added 0.25 g of sodium bicarbonate and 0.3 g of ethyl chloroformate.

It is heated to reflux for 16 h. It is concentrated to dryness and the residue is treated with 30 ml of water and extracted with 3×50 ml of dichloromethane-ethanol 90/10. The organic phase is dried and concentrated to a volume of 20 ml. The precipitated solid is filtered and 0.3 g of pure product is obtained.

¹H-RMN (DSMO-d₆, 200 MHz, δ (ppm)): 8.70 (s, 1H); 7.98 (d, 1H); 7.64 (d, 1H); 7.56 (dd, 1H); 7.50 (t, 1H, NH); 7.30-7.10 (s.c., 2H); 4.80-4.64 (m, 1H); 4.14 (t, 1H); 4.02 (c, 2H); 3.96-3.70 (s.c., 2H); 3.60-3.10 (s.c., 10H); 1.42-1.10 (s.c., 4H); 1.17 (t, 3H).

### Example 12

### 1-cyclopropyl-6-fluoro-7-{4-[2-fluoro-4-(5-(S)-{[3-(4-fluoro-phenyl)-acryloylamino]-methyl}-2-oxo-oxazolidin-3-yl)-phenyl]-piperazin-1-yl}-4-oxo-1,4-dihydro-quinoline-3-carboxylic Acid

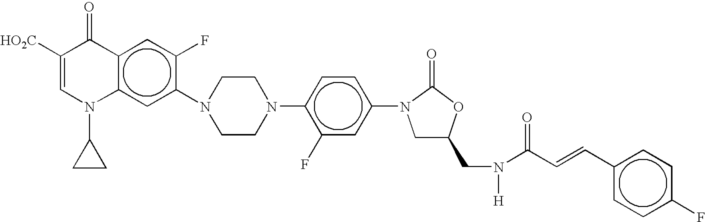

To 0.6 g (1.1 mmol) of the product of Reference Example No. 38 in 20 ml of dry dichloromethane are added 0.17 ml (1.22 mmol) of triethylamine and 0.3 g (1.33 mmol) of 4-fluorocinnamoyl chloride.

The reaction is maintained at room temperature for 16 h, then concentrated to dryness and the residue is chromatographed on silica gel.

Elution with dichloromethane-ethanol 95/5 yields 0.3 g of pure product.

¹H-RMN (DSMO-d₆, 200 MHz, δ (ppm)): 8.70 (s, 1H); 8.58 (t, 1H, NH); 7.96 (d, 1H); 7.70-7.58 (s.c., 4H); 7.44 (d, 1H); 7.30-7.10 (s.c., 4H); 6.64 (d, 1H); 4.90-4.76 (m, 1H); 4.16 (t, 1H); 3.92-3.70 (s.c., 2H); 3.64-3.10 (s.c., 10H); 1.42-1.10 (s.c., 4H)

### Example 13

### 1-cyclopropyl-7-[4-(4-{5-(S)-[(3-ethyl-thioureido)-methyl]-2-oxo-oxazolidin-3-yl}-2-fluoro-phenyl)-piperazin-1-yl]-6-fluoro-4-oxo-1,4-dihydro-quinoline-3-carboxylic Acid

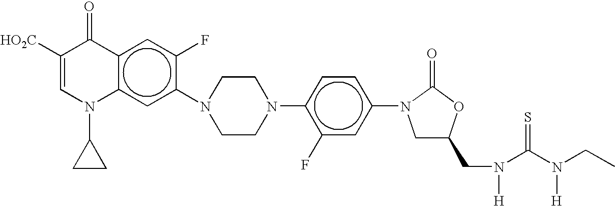

Following the procedure described in Example No. 9, replacing the methylisothiocyanate by ethylisothiocyanate, the product of the title is obtained.

¹H-RMN (DSMO-d₆, 200 MHz, δ (ppm)): 15.06 (s.a., 1H); 8.70 (s, 1H); 7.98-7.50 (m, 4H,); 7.30-7.10 (s.c., 2H); 4.95-4.80 (m, 1H); 4.16 (t, 1H); 4.00-3.70 (s.a., 4H); 3.60-3.10 (m., 10H); 1.44-1.16 (s.c., 4H).; 1.02 (t., 3H).

### Example 14

### 1-(2,4-difluoro-phenyl)-6-fluoro-7-(4-{2-fluoro-5-[5-(R)-(1-(R,S)-hydroxy-prop-2-inyl)-2-oxo-oxazolidin-3-yl]-phenyl}piperazin-1-yl)-4-oxo-1,4-dihydro-[1,8]naphthyridine-3-carboxylic Acid Ethyl Ester

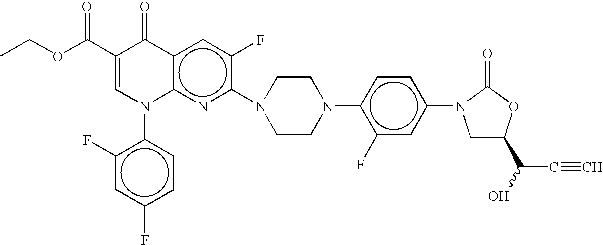

To 0.32 g (1 mmol) of the product of Reference Example No. 30 in 10 ml of pyridine are added 0.42 g (1 mmol) of 7-chloro-1-(2,4-difluorophenyl)-6-fluoro-4-oxo-1,4-dihydro-[1,8]naphthyridine-3-carboxylic acid ethyl ester (ACROS) and 0.28 ml of triethylamine. The reaction is maintained at room temperature for 48 h. It is concentrated to dryness and the residue is chromatographed on silica gel.

Elution with dichloromethane/ethanol/ammonium hydroxide 95/5/1% yields 0.436 g (66%) of the product of the title.

¹H-RMN (CDCl₃, 200 MHz, δ (ppm)): 8.42 (s, 1H); 8.15 (d, 1H); 7.40 (m, 2H); 7.10 (m, 3H); 6.90 (t, 1H) 4.75 (m, 1H); 4.70 (m, 1H); 4.38 (c, 2H); 4.10 (m, 2H); 3.70 (m, 4H); 3.04 (m, 4H); 2.50 (m, 1H); 1.40 (t, 3H).

### Example 15

### 7-(4-{4-[5-(S)-(acetylamino-methyl)-2-oxo-oxazolidin-3-yl]-2-fluoro-phenyl}-piperazin-1-yl)-1-(2,4-difluoro-phenyl)-6-fluoro-4-oxo-1,4-dihydro-[1,8]naphthyridine-3-carboxylic Acid Ethyl Ester

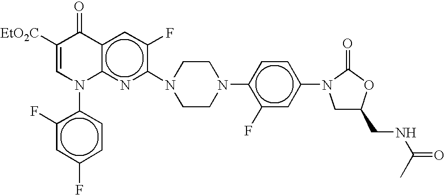

Following the procedure of the previous example and using N-[3-(3-Fluoro-4-piperazin-1-yl-phenyl)-2-oxo-oxazolidin-5-(S)-ylmethyl]-acetamid oxazolidin-5-(S)-ylmethyl]-acetamide (obtained according to U.S. Pat. No. 5,547,950) the product of the title is obtained.

¹H-RMN (CDCl₃, 200 MHz, δ (ppm)): 8.41 (s, 1H); 8.15 (d, 1H); 7.42 (dd, 1H); 7.16-6.80 (s.c., 5H); 6.41 (t, 1H, NH); 4.84-4.70 (m, 1H); 4.39 (c, 2H); 4.02 (t, 1H); 4.80-4.60 (s.c., 7H); 3.10-2.95 (s.a., 4H); 2.02 (s, 3H); 1.40 (t, 3H).

### Example 16

### 7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-[1,8]Naphthyridine-3-carboxylic Acid Ethyl Ester

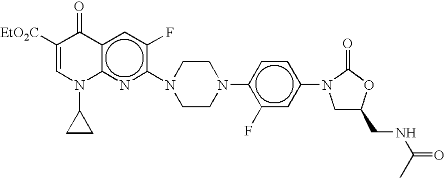

Following the procedure described in example 14 and using N-[3-(3-Fluoro-4-piperazin-1-yl-phenyl)-2-oxo-oxazolidin-5-(S)-ylmethyl]-acetamide (obtained according to U.S. Pat. No. 5,547,950) and 7-chloro-1-cyclopropyl-6-fluoro-4-oxo-1,4-dihydro-[1,8]naphthyridine-3-carboxylic acid ethyl ester (EP 0187376B1) the product of the title is obtained.

¹H-RMN (CDCl₃, 200 MHz, δ (ppm)): 8.52 (s, 1H); 8.11 (d, 1H); 7.48 (dd, 1H); 7.08 (m, 1H); 6.94 (t, 1H); 6.74 (t, 1H, NH); 4.79 (m, 1H); 4.37 (c, 2H); 4.01 (m, 5H); 3.76 (m, 1H); 3.66 (m, 2H); 3.53 (m, 1H); 3.20 (m, 4H); 2.04 (s, 3H); 1.40 (t, 3H); 1.23 (m, 2H); 1.05 (m, 2H).

### Example 17

### 7-(4-{4-[5-(S)-(acetylamino-methyl)-2-oxo-oxazolidin-3-yl]-2-fluoro-phenyl}-piperazin-1-yl)-6,8-difluoro-1-(2-fluoro-ethyl)-4-oxo-1,4-dihydro-quinoline-3-carboxylic Acid Ethyl Ester

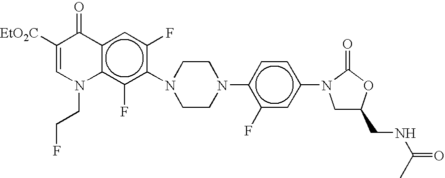

Following a procedure analogous to the previous ones and replacing the derivative of naphthyridine by 6,7,8-trifluoro-1-(2-fluoro-ethyl)-4-oxo-1,4-dihydro-quinoline-3-carboxylic acid ethyl ester, the product of the title is obtained.

¹H-RMN (DMSO-d₆, 200 MHz, δ (ppm)) 8.59 (s, 1H); 8.30 (t, 1H, NH); 7.79 (d, 1H); 7.50 (d, 1H); 7.30-7.00 (s.c., 2H); 5.05-4.60 (s.c., 5H); 4.21 (c, 2H); 4.15 (t, 1H); 3.80-3.00 (s.c., 11H); 1.82 (s, 3H); 1.27 (t, 3H).

### Example 18

### 1-(2,4-Difluoro-phenyl)-6-fluoro-7-(4-{2-fluoro-4-[5-(S)-(isoxazol-3-ylaminomethyl)-2-oxo-oxazolidin-3-yl]-phenyl}-piperazin-1-yl)-4-oxo-1,4-dihydro-[1,8]naphthyridine-3-carboxylic Acid Ethyl Ester

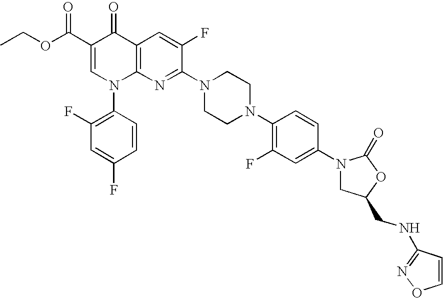

Following the procedure of example 14 and replacing the product of Reference Example No. 30 by the product of Reference Example No. 25 1-(2,4-difluoro-phenyl)-6-fluoro-7-{4-[2-fluoro-4-(5-(R)-{[isoxazol-3-yl-(2,2,2-trichloro-ethoxycarbonyl)-amino]-methyl}-2-oxo-oxazolidin-3-yl)-phenyl]-piperazin-1-yl}-4-oxo-1,4-dihydro-[1,8]naphthyridine-3-carboxylic acid ethyl ester is obtained. To 500 mg thereof, dissolved in 10 ml of tetrahydrofuran is added 5 ml of water, 5 ml of glacial acetic acid and 700 mg of powdered zinc. After stirring for 3 h at room temperature it is filtered over decalite and the filtering liquids concentrated and chromatographed on silica gel. Elution with dichloromethane/ethanol/ammonium hydroxide 98/2/02% yields 247 mg of the product of the title.

¹H-RMN (CDCl₃, 200 MHz, δ (ppm)): 8.41 (s, 1H); 8.15 (d, 1H); 8.07 (d, 1H); 7.45 (m, 2H); 7.05 (m, 3H); 6.85 (t, 1H); 5.85 (s, 1H); 4.95 (m, 1H); 4.50 (m, 1H); 4.38 (c, 2H); 4.05 (t, 1H); 3.80, (m, 2H); 3.68 (m, 4H) 3.03 (m, 4H); 1.39 (t, 3H).

### Example 19

### 1-(2,4-difluoro-phenyl)-6-fluoro-7-(4-{2-fluoro-4-[5-(R)-(1-hydroxy-prop-2-inyl)-2-oxo-oxazolidin-3-yl]-phenyl}-piperazin-1-yl)-4-oxo-1,4-dihydro-[1,8]Naphthyridine-3-carboxylic Acid

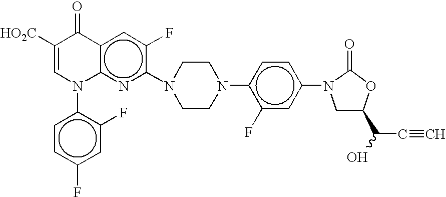

To 0.436 g (0.6 mmol) of the product of example 14 in 5 ml of ethanol and 5 ml of water is added 1.32 ml of sodium hydroxyde 1N. It is heated at 50° C. for 3 h. 1.32 ml of HCl 1N is added and it is concentrated to dryness. The residue is chromatographed on silica gel. Elution with dichloromethane/ethanol/acetic acid 95/5/0.5% yields 0.287 g (75%) of the product of the title.

¹H-RMN (CDCl₃, 200 MHz, δ (ppm)): 8.68 (s, 1H); 8.15 (d, 1H); 7.60-7.27 (m, 2H); 7.20-7.00 (m, 3H); 6.90 (t, 1H); 4.75 (m, 1H); 4.30-4.00 (m, 2H); 3.80 (m, 4H); 3.28 (dd, 1H); 3.20 (m, 1H); 2.50 (d, 1H).

### Example 20

### 7-(4-{4-[5-(S)-(acetylamino-methyl)-2-oxo-oxazolidin-3-yl]-2-fluoro-phenyl}-piperazin-1-yl)-1-(2,4-difluoro-phenyl)-6-fluoro-4-oxo-1,4-dihydro-[1,8]Naphthyridine-3-carboxylic Acid

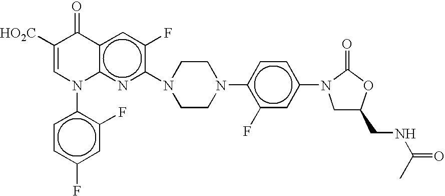

Following the procedure described in the previous example and using the product described in Example No. 15 the product of the title is obtained.

¹H-RMN (DMSO-d₆, 200 MHz, δ (ppm)) 8.90 (s, 1H), 8.27 (t, 1H); 8.22 (d, 1H); 7.95-7.80 (m, 1H); 7.80-7.60 (m, 1H); 7.50 (d, 1H); 7.45-7.30 (m, 1H); 7.25-7.00 (s.c., 2H); 4.80-4.62 (m, 1H); 4.12 (t, 1H); 3.80-2.95 (s.c., 11H); 1.84 (s, 3H).

### Example 21

### 7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-[1,8]Naphthyridine-3-carboxylic Acid

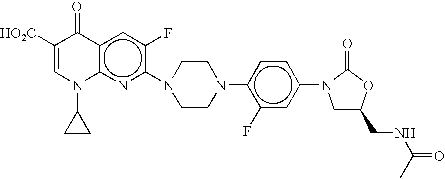

From the product of Example No. 16 and following the procedure described above the product of the title is obtained.

¹H-RMN (CDCl₃, 200 MHz, δ (ppm)): 8.74 (s, 1H); 8.12 (m, 1H); 8.10 (d, 1H); 7.50 (m, 1H); 7.12 (m, 1H); 6.95 (t, 1H); 4.79 (m, 1H); 4.10 (m, 4H); 4.05 (m, 1H); 3.89 (m, 1H); 3.67 (m, 1H); 3.58 (m, 2H); 3.24 (m, 4H); 2.00 (s, 3H); 1.30 (m, 2H); 1.15 (m, 2H).

### Example 22

### 7-(4-{4-[5-(S)-(acetylamino-methyl)-2-oxo-oxazolidin-3-yl]-2-fluoro-phenyl}-piperazin-1-yl)-6,8-difluoro-1-(2-fluoro-ethyl)-4-oxo-1,4-dihydro-quinoline-3-carboxylic Acid

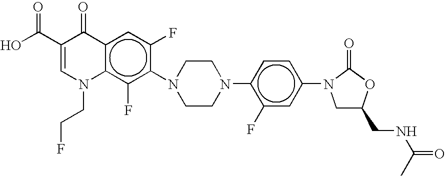

From the product of Example No. 17 and following the procedure described in Example No. 19, the product of the title is obtained.

¹H-RMN (DMSO-d₆, 200 MHz, δ (ppm)): 8.84 (s, 1H); 8.26 (t, 1H, NH); 7.92 (d, 1H); 7.56 (d, 1H); 7.35-7.05 (s.c., 2H); 5.16-4.64 (s.c., 5H); 4.12 (t, 1H); 3.80-3.00 (s.c., 11H); 1.82 (s, 3H).

### Example 23

### 1-(2,4-Difluoro-phenyl)-6-fluoro-7-(4-{2-fluoro-4-[5-(S)-(isoxazol-3-ylaminomethyl)-2-oxo-oxazolidin-3-yl]-phenyl}-piperazin-1-yl)-4-oxo-1,4-dihydro-[1,8]naphthyridine-3-carboxylic Acid

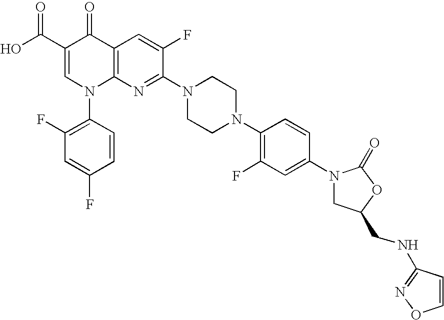

From the product of Example No. 18 and following procedure described in Example No. 19, the product of the title is obtained.

¹H-RMN (CDCl₃, 200 MHz, δ (ppm)): 8.69 (s, 1H) 8.15 (d, 1H); 8.06 (d, 1H); 7.45 (m, 2H); 7.10 (m, 3H); 6.90 (t, 1H); 5.90 (s, 1H); 4.95 (m, 1H); 4.50 (m, 1H); 4.06 (t, 1H); 4.00-3.50 (m, 6H); 3.05 (m, 4H).

### Example 24

### 1-ethyl-6,8-difluoro-7-[4-(2-fluoro-4-{5-[(3-methyl-thioureido)-methyl]-2-oxo-oxazolidin-3-yl}-phenyl)-piperazin-1-yl]-4-oxo-1,4-dihydro-quinoline-3-carboxylic Acid

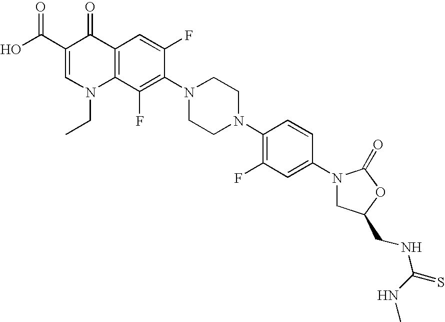

To 2 g (6.7 mmol) of 1-ethyl-6,7,8-trifluoro-4-oxo-1,4-dihydro-quinoline-3-carboxylic acid ethyl ester in 40 ml of N-methyl-2-pyrrolidone are added 3.1 g (6.7 mmol) of the product of Reference Example No. 33 and 1.85 ml of triethylamine. The reaction is heated at 100° C. for 48 h.

The solvent is distilled under vacuum and the residue is chromatographed on silica gel. Elution with dichloromethane/ethanol 90/10 yields 7-{4-[4-(5-(S)-aminomethyl-2-oxo-oxazolidin-3-yl)-2-fluoro-phenyl]-piperazin-1-yl}-1-ethyl-6,8-difluoro-4-oxo-1,4-dihydro-quinoline-3-carboxylic acid ethyl ester. From said product and by following procedure described in Example No. 19, the product of the title is obtained.

IR: 3380 cm⁻¹ 1750 cm⁻¹ 1620 cm⁻¹ 1510 cm⁻¹

### Example 25

### 1-cyclopropyl-6-fluoro-7-[4-(2-fluoro-4-{2-oxo-5-(S)-[(3-propyl-thioureido)-methyl]-oxazolidin-3-yl}-phenyl)-piperazin-1-yl]-4-oxo-1,4-dihydro-quinoline-3-carboxylic Acid

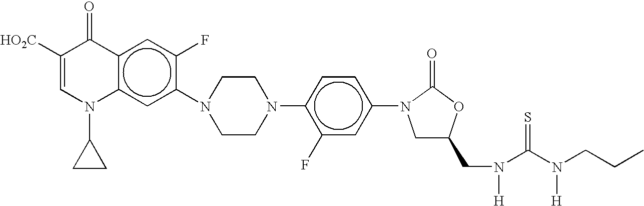

Following the procedure described in Example No. 9, replacing the methylisothiocyanate by propylisothiocyanate, the product of the title is obtained.

¹H-RMN (DSMO-d₆, 200 MHz, δ (ppm)): 8.70 (s, 1H); 7.92 (d., 1H), 7.90-7.70 (m, 2H, NH); 7.70-7.50 (m., 2H); 7.30-7.10 (m., 2H); 4.95-4.80 (m, 1H); 4.16 (t, 1H); 4.00-3.70 (s.a., 4H); 3.60-3.10 (m., 10H); 1.60-1.16 (s.c., 6H).; 0.84 (t., 3H).

### Example 26

### 1-cyclopropyl-6-fluoro-7-[4-{2-fluoro-4-[5-(S)-(methanesulfonylamino-methyl)-2-oxo-oxazolidin-3-yl]-phenyl}-piperazin-1-yl)-4-oxo-1,4-dihydro-quinoline-3-carboxylic Acid

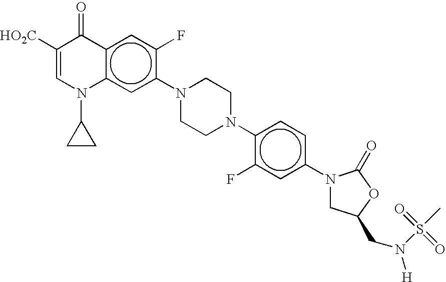

Following the procedure described in Example No. 9, replacing the methylisothiocyanate by methanesulphonylchloride, the product of the title is obtained.

¹H-RMN (DSMO-d₆, 200 MHz, δ (ppm)): 15.00 (s.a., 1H); 8.70 (s, 1H); 7.96 (d., 1H), 7.76-7.42 (m, 3H); 7.30-7.10 (m., 2H); 4.90-4.76 (m, 1H); 4.18 (t, 1H); 4.00-3.20 (m., 12H); 2.98 (s, 3H); 1.44-1.16 (m., 4H).

### Example 27

### 7-(4-{4-[5-(S)-(Acetylamino-methyl)-2-oxo-oxazolidin-3-yl]-phenyl}-piperazin-1-yl)-1-ethyl-6,8-fluoro-4-oxo-1,4-dihydro-quinoline-3-carboxylic Acid Ethyl Ester

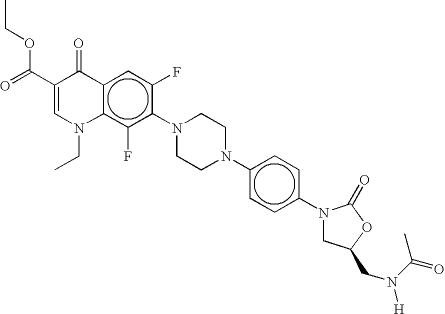

Following the procedure described in Example No. 14, using the product obtained in Reference Example No. 26 and 1-ethyl-6,7,8-trifluoro-4-oxo-1,4-dihydro-quinoline-3-carboxylic acid ethyl ester (obtained by esterification of he corresponding acid, described in GB 2057440).

¹H-RMN (DSMO-d₆, 200 MHz, δ (ppm)): 8.62 (s, 1H); 8.30 (t, 1H, NH); 7.80 (d., 1H), 7.42 (d, 2H); 7.04 (d., 2H); 4.84-4.64 (m, 1H); 4.60-4.40 (s.a., 2H); 4.26 (c, 2H); 4.16 (t, 1H); 3.78 (t, 1H); 3.60-3.20 (m., 10H); 1.90 (s, 3H); 1.44 (t, 3H); 1.30 (t., 3H).

### Example 28

### 1-cyclopropyl-6-fluoro-7-[4-(2-fluoro-4-{2-oxo-5-(S)-[(2,2,2-trifluoro-acetylamino)-methyl]-oxazolidin-3-yl}-phenyl)-piperazin-1-yl]-4-oxo-1,4-dihydro-quinoline-3-carboxylic Acid

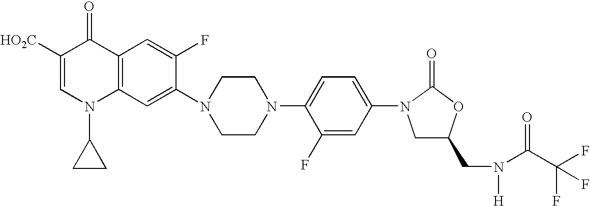

Following the procedure described in Example No. 9, replacing the methylisothiocyanate by trifluoroacetic anhydride, the product of the title is obtained.

¹H-RMN (DSMO-d₆, 200 MHz, δ (ppm)): 15.06 (s.a., 1H); 9.92 (s.a. , 1H, NH); 8.70 (s, 1H); 7.95 (d, 1H,); 7.70-7.50 (m, 2H); 7.30-7.10 (s.c., 2H); 4.95-4.80 (m, 1H); 4.20 (t, 1H); 4.00-3.80 (s.a. , 2H); 3.60-3.20 (m., 10H); 1.44-1.16 (m., 4H).

### Example 29

### 7-(4-{4-[5-(S)-(benzoylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic Acid

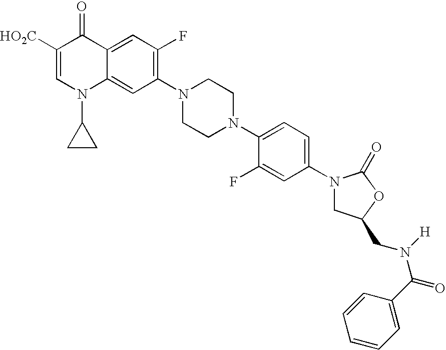

Following the procedure described in Example No. 9, replacing the methylisothiocyanate by benzoyl chloride, the product of the title is obtained.

¹H-RMN (DSMO-d₆, 200 MHz, δ (ppm)): 15.20 (s.a., 1H); 8.90 (t, 1H, NH); 8.70 (s, 1H); 8.00-7.85 (m., 3H) 7.76-7.42 (m, 5H); 7.30-7.10 (m. , 2H); 4.96-4.80 (m, 1H) 4.20 (t, 1H); 4.00-3.20 (m., 12H); 1.44-1.16 (m., 4H).

### Example 30

### 7-(4-{4-[5-(S)-(Acetylamino-methyl]-2-oxo-oxazolidin-3-yl]2-fluoro-phenyl)-piperazin-1-yl)-1-cyclopropyl-6-fluoro-4-oxo-1,4-dihydro-quinoline-3-carboxylic Acid Methyl Ester

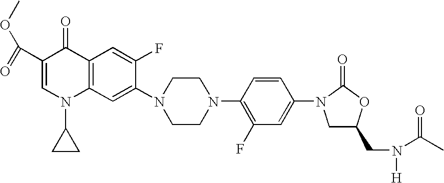

To 1 g (1.7 mmol) of the product of Example 1 in 30 ml of methanol cooled to 0° C. is added dropwise 0.37 ml (5.2 mmol) of thionyl chloride. When the addition is finished it is heated to reflux for 48 hours. It is concentrated to dryness and the residue is chromatographed on silica gel. Elution with dichloromethane/methanol/acetic acid 90/10/1 yields the product of the title as hydrochloride.

The product thus obtained is dissolved in dichloromethane/methanol 90/10 and washed with saturated solution of sodium bicarbonate. The organic phase is dried and concentrated to yield the product of the title in the form of free base.

¹H-RMN (DSMO-d₆, 200 MHz, δ (ppm)): 8.50 (s, 1H); 8.25 (s.a., 1H, NH); 7.92 (d., 1H), 7.64-7.50 (m, 2H); 7.30-7.10 (m., 2H); 4.90-4.70 (m, 1H); 4.16 (t, 1H); 3.90-3.60 (m., 5H); 3.60-3.20 (m., 10H); 1.86 (s., 1H); 1.45-1.10 (m., 4H).

### Example 31

### 9-[3-(S)-({4-[5-(S)-(Acetylamino-methyl)-2-oxo-oxazolidin-3-yl]-2-fluoro-phenyl}-methyl-amino)-pyrrolidin-1-yl]-8-fluoro-3-(S)-methyl-6-oxo-2,3-dihydro-6H-1-oxa-3a-aza-phenalene-5-carboxylic Acid

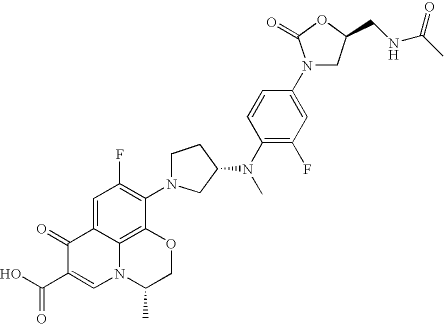

Following the procedure described in Example 3 and starting with the corresponding chelate obtained by reaction of N-{3-(S)-[3-Fluoro-4-(methyl-pyrrolidin-3-yl-amino)-phenyl]-2-oxo-oxazolidin-5-(S)-ylmethyl}-acetamide (obtained following the procedure for the obtention of Reference Example No. 27, but replacing 3(R,S)-aminopyrrolidine by 3-(S)-aminopyrrolidine) and 8,9-Difluoro-3-(S)-methyl-6-oxo-2,3-dihydro-6H-1-oxa-3a-aza-phenalene-5-carboxylic acid boron difluoride chelate the product of the title is obtained.

¹H-RMN (DSMO-d₆, 200 MHz, δ (ppm)): 8.92 (s, 1H); 8.24 (t, 1H, NH); 7.60-7.40 (m, 2H); 7.30-7.10 (m, 2H); 4.95-4.80 (m, 1H); 4.80-4.60 (m, 1H); 4.52 (d, 1H); 4.30 (d, 1H); 4.10 (t, 1H), 4.00-3.30 (m, 8H); 2.74 (s, 3H); 2.20-1.80 (m, 2H); 1.84 (s, 3H); 1.42 (d, 3H).

[α]²⁵_{D}=−34° (c 0.5, CH₂Cl₂/MeOH 9/1)

### Example 32

### 9-[3-(S)-({4-[5-(S)-(Acetylamino-methyl)-2-oxo-oxazolidin-3-yl]-2-fluoro-phenyl}-methyl-amino)-pyrrolidin-1-yl]-8-fluoro-3-(R)-methyl-6-oxo-2,3-dihydro-6H-1-oxa-3a-aza-phenalene-5-carboxylic Acid

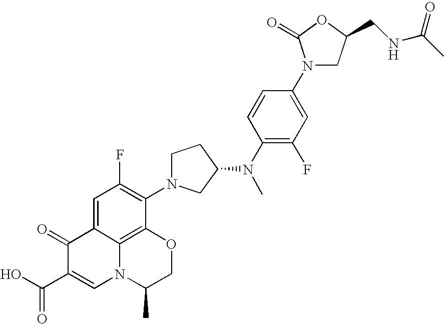

Following the procedure described in Example 3 and starting with the corresponding chelate obtained by reaction of N-{3-(S)-[3-Fluoro-4-(methyl-pyrrolidin-3-yl-amino)-phenyl]-2-oxo-oxazolidin-5-(S)-ylmethyl}-acetamide (obtained following the procedure for the obtention of Reference Example No. 27, but replacing 3(R,S)-aminopyrrolidine by 3-(S)-aminopyrrolidine) and 8,9-Difluoro-3-(R)-methyl-6-oxo-2,3-dihydro-6H-1-oxa-3a-aza-phenalene-5-carboxylic acid boron difluoride chelate (obtained according to Shohgo Atarashi et al., Chem. Pharm. Bull. (1987), 35 (5), 1896-1902) the product of the title is obtained.

¹H-RMN (DSMO-d₆, 200 MHz, δ (ppm)): 8.90 (s, 1H); 8.24 (t, 1H,. NH); 7.60-7.40 (m, 2H); 7.36-7.10 (m, 2H); 4.95-4.80 (m, 1H); 4.80-4.60 (m, 1H); 4.54 (d, 1H); 4.24 (d, 1H); 4.10 (t, 1H), 4.00-3.30 (m, 8H); 2.74 (s, 3H); 2.20-1.80 (m, 2H); 1.84 (s, 3H); 1.42 (d, 3H).

[α]²⁵_{D}=+66.4° (c 0.5, CH₂Cl₂/MeOH 9/1)

### Example 33

### 9-[3-(R)-({4-[5-(S)-(Acetylamino-methyl)-2-oxo-oxazolidin-3-yl]-2-fluoro-phenyl}-methyl-amino)-pyrrolidin-1-yl]-8-fluoro-3-(S)-methyl-6-oxo-2,3-dihydro-6H-1-oxa-3a-aza-phenalene-5-carboxylic Acid

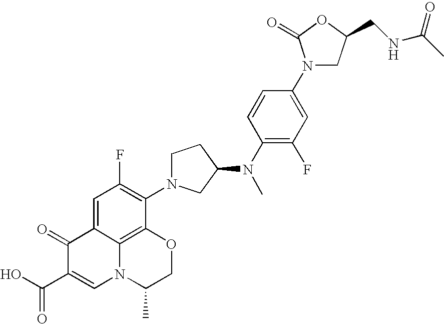

Following the procedure described in Example 3 and starting with the corresponding chelate obtained by reaction of N-{3-(R)-[3-Fluoro-4-(methyl-pyrrolidin-3-yl-amino)-phenyl]-2-oxo-oxazolidin-5-(S)-ylmethyl}-acetamide (obtained following the procedure for the obtention of Reference Example No. 27, but replacing 3(R,S)-aminopyrrolidine by 3-(R)-aminopyrrolidine) and 8,9-Difluoro-3-(S)-methyl-6-oxo-2,3-dihydro-6H-1-oxa-3a-aza-phenalene-5-carboxylic acid boron difluoride chelate the product of the title is obtained.

¹H-RMN (DSMO-d₆, 200 MHz, δ (ppm)): 8.92 (s, 1H); 8.24 (t, 1H, NH); 7.60-7.40 (m, 2H); 7.36-7.10 (m, 2H); 4.95-4.80 (m, 1H); 4.80-4.60 (m, 1H); 4.56 (d, 1H); 4.26 (d, 1H); 4.10 (t, 1H) , 4.02-3.30 (m, 8H); 2.76 (s, 3H) 2.20-1.80 (m, 2H); 1.82 (s, 3H); 1.40 (d, 3H).

[a]²⁵_{D}=−80.6° (c 0.5, CH₂Cl₂/MeOH 9/1)

### Example 34

### 9-[3-(R)-({4-[5-(S)-(Acetylamino-methyl)-2-oxo-oxazolidin-3-yl]-2-fluoro-phenyl}-methyl-amino)-pyrrolidin-1-yl]-8-fluoro-3-(R)-methyl-6-oxo-2,3-dihydro-6H-1-oxa-3a-aza-phenalene-5-carboxylic Acid

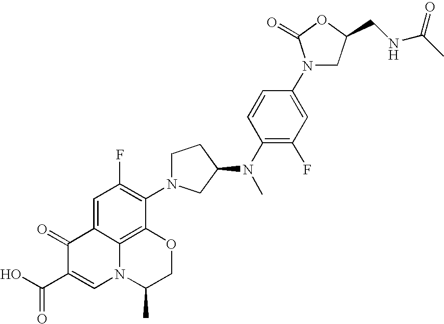

Following the procedure described in Example 3 and starting with the corresponding chelate obtained by reaction of N-{3-(R)-[3-Fluoro-4-(methyl-pyrrolidin-3-yl-amino)-phenyl]-2-oxo-oxazolidin-5-(S)-ylmethyl}-acetamide (obtained following the procedure for the obtention of Reference Example No. 27, but replacing 3(R,S)-aminopyrrolidine by 3-(R)-aminopyrrolidine) and 8,9-Difluoro-3-(R)-methyl-6-oxo-2,3-dihydro-6H-1-oxa-3a-aza-phenalene-5-carboxylic acid boron difluoride chelate (obtained according to Shohgo Atarashi et al., Chem. Pharm. Bull. (1987), 35 (5), 1896-1902) the product of the title is obtained.

¹H-RMN (DSMO-d₆, 200 MHz, δ (ppm)): 8.90 (s, 1H); 8.24 (t, 1H, NH); 7.60-7.40 (m, 2H); 7.36-7.10 (m, 2H); 4.95-4.80 (m, 1H); 4.80-4.60 (m, 1H); 4.54 (d, 1H); 4.30 (d, 1H); 4.10 (t, 1H), 4.00-3.30 (m, 8H); 2.72 (s, 3H); 2.20-1.80 (m, 2H); 1.84 (s, 3H); 1.42 (d, 3H).

[α]²⁵_{D}=+18° (c 0.5, CH₂Cl₂/MeOH 9/1)

### Example 35

### 1-Cyclopropyl-6-fluoro-7-(4-{2-fluoro-4-[5-(S)-(isoxazol-3-ylaminomethyl)-2-oxo-oxazolidin-3-yl]-phenyl}-piperazin-1-yl)-4-oxo-1,4-dihydro-[1,8]naphthyridine-3-carboxylic Acid

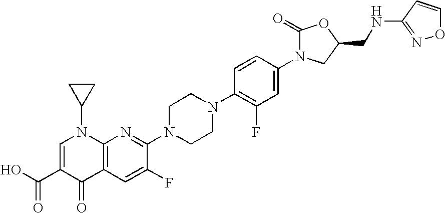

Following the procedure described in Example 14 and starting with the corresponding product obtained by reaction of the compound in reference Example 25 N-deprotected and 7-Chloro-1-cyclopropyl-6-fluoro-4-oxo-1,4-dihydro-[1,8]naphthyridine-3-carboxylic acid the product of the title is obtained.

¹H-RMN (DSMO-d₆, 200 MHz, δ (ppm)): 13.2 (s, 1H); 8.61 (s, 1H); 8.40 (s, 1H); 8.10 (d, 1H); 7.50 (d, 1H); 7.10 (m, 2H); 6.55 (t, 1H); 5.98 (s, 1H); 4.85 (m, 1H); 4.04 (m, 5H); 3.75 (m, 2H); 3.40 (m, 2H); 3.17 (m, 4H); 1.2 (m, 4H).

### Examples of Pharmacological Results

### Description of the Methods Used for Evaluation of the Pharmacological Properties

The antibacterial activity of the new synthesised compounds on the various strains of the bacterial species was implemented using the technique of microdilution in culture broth according to the regulations of the National Committee for Clinical Laboratory Standards (NCCLS), (NCCLS. 1993. Methods for dilution antimicrobial susceptibility tests for bacteria that grow aerobically. Approved standard M7-A3. NCCLS, Vilanova. Pa., and NCCLS. 1993. Methods for dilution antimicrobial susceptibility tests for anaerobic bacteria that grow aerobically. Approved standard M1-A3. NCCLS, Vilanova. Pa.).

The inoculum used was 5×10⁵ UFC/ml following dilution of the cultures overnight in the exponential phase of bacterial growth.

The MIC expressed in mg/l was defined as the minimum concentration of antibiotic which inhibited any visible growth.

Linezolid was included as comparative compound.

The compounds were tested on the strains of G(+) and G(−) bacteria set out in Table 1, in which:

TABLE 1
Antibacterial activity on hospital strains (resistant) of Gram (+)
and Gram (−) bacteria

| | G (+) strains | | | | | | Anaerobic | G (−) strains | | |
|---|---|---|---|---|---|---|---|---|---|---|
| PRODUCT | A | B | C | D | E | F | G | H | I | J |
| Linezolid | 2 | 2 | 1 | 2 | 1 | 1 | 2 | >64 | 16 | 16 |
| EXAMP. 1 + 8 | 0.25 | 0.125 | <0.125 | 0.125 | 0.125 | <0.125 | 0.25 | 8 | 0.25 | 0.25 |
| EXAMP. 3 | <0.125 | <0.125 | <0.125 | <0.125 | <0.125 | <0.125 | 0.5 | 64 | 4 | 0.5 |
| EXAMP. 4 | 0.25 | 0.25 | <0.125 | <0.125 | <0.125 | <0.125 | 0.5 | 16 | 2 | 1 |
| EXAMP. 5 | 0.5 | 0.5 | 0.5 | 0.5 | 0.25 | 0.5 | 2 | 4 | 1 | 1 |
| EXAMP. 6 | 2 | 1 | 1 | 0.5 | 0.5 | 1 | 16 | 2 | <0.125 | 0.25 |
| EXAMP. 7 | 0.25 | 0.5 | 0.25 | 0.5 | 0.25 | 0.25 | 2 | 8 | 4 | 1 |
| EXAMP. 9 | <0.125 | <0.125 | <0.125 | <0.125 | <0.125 | <0.125 | 0.5 | >64 | 1 | 0.25 |
| EXAMP. | 1 | 2 | 1 | 1 | 0.25 | 1 | 4 | 64 | 4 | 2 |
| 10 | | | | | | | | | | |
| EXAMP. | 0.5 | 0.5 | 0.5 | 1 | 0.125 | 0.5 | 1 | 32 | 4 | 1 |
| 11 | | | | | | | | | | |
| EXAMP. | 4 | 2 | 1 | 1 | 2 | 1 | 8 | >64 | >64 | 8 |
| 16 | | | | | | | | | | |
| EXAMP. | 2 | 2 | 0.5 | 0.5 | 1 | 1 | 4 | >64 | 64 | 4 |
| 17 | | | | | | | | | | |
| EXAMP. | 4 | 8 | 4 | 8 | 4 | 8 | >64 | 32 | 1 | 2 |
| 19 | | | | | | | | | | |
| EXAMP. | 1 | 2 | 1 | 1 | 0.50 | 1 | >64 | >64 | 2 | 4 |
| 20 | | | | | | | | | | |
| EXAMP. | 0.25 | <0.125 | <0.125 | <0.125 | <0.125 | <0.125 | 0.25 | 64 | 2 | 0.5 |
| 21 | | | | | | | | | | |
| EXAMP. | <0.125 | <0.125 | 0.25 | <0.125 | <0.125 | <0.125 | 0.5 | 64 | 2 | 0.5 |
| 22 | | | | | | | | | | |

A: S. aureus resistant to meticillin
B: E. faecalis resistant to vancomycin
C: S. pneumoniae resistant to penicillin
D: S. agalactiae
E: S. epidermidis
F: S. pyogenes
G: B. fragilis
H: E. coli
I: H. influenzae
J: M. Catarrahalis.

1. Compound of general formula (I): 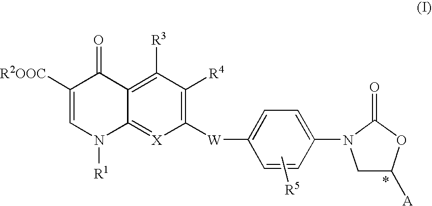
wherein:
X: CR⁶ or N;
R¹: alkyl C₁-C₄, cycloalkyl C₃-C₆, alkenyl C₂-C₄, 2-hydroxyethyl, 2-fluoroethyl, or phenyl optionally substituted by 1 or 2 atoms of fluorine;
R²: H, alkyl C₁-C₄ or phenyl;
R³: H, halogen, alkyl C₁-C₄, or alkoxy C₁-C₄, amino;
R⁴: H or halogen;
R⁶: H, halogen, alkyl C₁-C₄, haloalkoxy C₁-C₄, or else R¹and R⁶ together form a bridge of structure 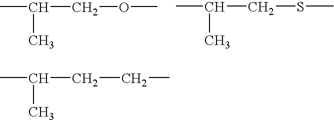
R⁵: H, halogen, OCH₃, alkoxy C₁-C₄, alkyl C₁-C₄, or haloalkyl C₁-C₄;
A: —CH₂—NH—R⁷, —CHOH—C≡CH;
wherein
R⁷: isoxazol, —CO—R⁸, —CS—R⁸, —CS—OR⁸, —COOR⁸, —CONHR⁸, —CSNHR⁸, —SO₂—R⁸ or 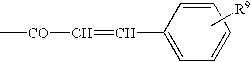
wherein
R⁸: alkyl C₁-C₄, haloalkyl C₁-C₄, alkenyl C₂-C₄, aryl, alkyl C₁-C₄ substituted by an alkoxy group C₁-C₄, carboxyalkyl C₁-C₄, cyano, or amino;
R⁹: H, alkyl C₁-C₄, alkenyl C₂-C₄, OH, alkoxy C₁-C₄, NR¹²R¹³, NO₂, halogen, or CO—R¹²;
R¹² and R¹³: independently, H or alkyl C₁-C₄;
W: 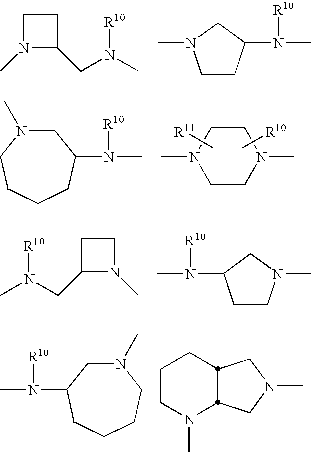
wherein
R¹⁰ and R¹¹ are independently H, or alkyl C₁-C₄;
a pharmaceutically acceptable salt or solvate, or any geometric isomer, optical isomer or mixture of isomers thereof in any proportion or polymorph thereof.

2. Compound according to claim 1, characterised in that R¹ is cyclopropyl, ethyl, 2-fluoroethyl, phenyl or difluorophenyl, or else R¹ and R⁶ together form a bridge of structure:

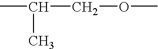

3. Compound according to claim 1, characterised in that R⁶ is H, CH₃, OCH₃, OCHF₂, F or Cl.

4. Compound according to claim 3, characterised in that R⁶ is H or F.

5. Compound according to claim 1, characterised in that R⁴ is F or Cl and R³ is H.

6. Compound according to claim 1, characterised in that W is 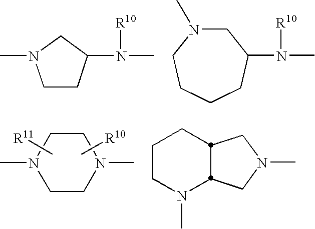
wherein R¹⁰ and R¹¹ are as defined in claim 1.

7. Compound according to claim 1, characterised in that the C5 of oxazolidinone ring has an (S) configuration when A=—CH₂—NH—R⁷ and (R) when A=—CHOH—C≡CH.

8. Compound according to claims 1 to 6, characterised in that it is selected from one of the following:
7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid
7-[3-({4-[5-(S)-(acetylamino-methyl)-2-oxo-oxazolidin-3-yl]-2-fluoro-phenyl}-methyl-amino) -azepan-1-yl]-1-cyclopropyl-6-fluoro-4-oxo-1,4-dihydro-quinoline-3-carboxylic acid
7-(4-{4-[5-(S)-(acetylamino-methyl)-2-oxo-oxazolidin-3-yl]-2-fluoro-phenyl}-piperazin-1-yl)-1-ethyl-6,8-difluoro-4-oxo-1,4-dihydro-quinoline-3-carboxylic acid
7-(4-{4-[5-(S)-(acetylamino-methyl)-2-oxo-oxazolidin-3-yl]-2-fluoro-phenyl}-piperazin-1-yl)-1-ethyl-6-fluoro-4-oxo-1,4-dihydro-quinoline-3-carboxylic acid
9-(4-{4-[5-(S)-(acetylamino-methyl)-2-oxo-oxazolidin-3-yl]-phenyl}-piperazin-1-yl) -8-fluoro-3-methyl-6-oxo-2,3-dihydro-6H-1-oxa-3a-aza-phenalen-5-carboxylic acid
9-[3-({4-[5-(S)-(acetylamino-methyl)-2-oxo-oxazolidin-3-yl]-2-fluoro-phenyl}-methyl-amino)-pyrrolidin-1-yl]-8-fluoro-3-methyl-6-oxo-2,3-dihydro-6H-1-oxa-3a-aza-phenalen-5-carboxylic acid
9-(4-{4-[5-(S)-(acetylamino-methyl)-2-oxo-oxazolidin-3-yl]-2-fluoro-phenyl}-piperazin-1-yl)-8-fluoro-3-methyl-6-oxo-2,3-dihydro-6H-1-oxa-3a-aza-phenalen-5-carboxylic
1-cyclopropyl-6-fluoro-7-[4-(2-fluoro-4-{5-(S)-[(3-methyl-thioureido)-methyl]-2-oxo-oxazolidin-3-yl}-phenyl)-piperazin-1-yl]-4-oxo-1,4-dihydro-quinoline-3-carboxylic acid
1-cyclopropyl-7-[4-(4-{5-(S)-[(3-ethyl-ureido)-methyl]-2-oxo-oxazolidin-3-yl}-2-fluoro-phenyl)-piperazin-1-yl]-6-fluoro-4-oxo-1,4-dihydro-quinoline-3-carboxylic acid
1-cyclopropyl-7-(4-{4-[5-(S)-(ethoxycarbonylamino-methyl)-2-oxo-oxazolidin-3-yl]-2-fluoro-phenyl}-piperazin-1-yl)-6-fluoro-4-oxo-1,4-dihydro-quinoline-3-carboxylic acid
1-cyclopropyl-6-fluoro-7-{4-[2-fluoro-4-(5-(S)-{[3-(4-fluoro-phenyl)-acryloylamino]-methyl}-2-oxo-oxazolidin-3-yl)-phenyl]-piperazin-1-yl}-4-oxo-1,4-dihydro-quinoline-3-carboxylic acid
1-cyclopropyl-7-[4-(4-{5-(S)-[(3-ethyl-thioureido)-methyl]-2-oxo-oxazolidin-3-yl}-2-fluoro-phenyl)-piperazin-1-yl]-6-fluoro-4-oxo-1,4-dihydro-quinoline-3-carboxylic acid
1-(2,4-difluoro-phenyl)-6-fluoro-7-(4-{2-fluoro-5-[5-(R)-(1-(R,S)-hydroxy-prop-2-inyl)-2-oxo-oxazolidin-3-yl]-phenyl}piperazin-1-yl)-4-oxo-1,4-dihydro-[1,8]naphthyridine-3-carboxylic acid ethyl ester
7-(4-{4-[5-(S)-(acetylamino-methyl)-2-oxo-oxazolidin-3-yl]-2-fluoro-phenyl}-piperazin-1-yl)-1-(2,4-difluoro-phenyl)-6-fluoro-4-oxo-1,4-dihydro-[1,8]naphthyridine-3-carboxylic acid ethyl ester
7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-[1,8]naphthyridine-3-carboxylic acid ethyl ester
7-(4-{4-[5-(S)-(acetylamino-methyl)-2-oxo-oxazolidin-3-yl]-2-fluoro-phenyl}-piperazin-1-yl)-6,8-difluoro-1-(2-fluoro-ethyl)-4-oxo-1,4-dihydro-quinoline-3-carboxylic acid ethyl ester
1-(2,4-Difluoro-phenyl)-6-fluoro-7-(4-{2-fluoro-4-[5-(S)-(isoxazol-3-ylaminomethyl)-2-oxo-oxazolidin-3-yl]-phenyl}-piperazin-1-yl)-4-oxo-1,4-dihydro-[1,8]naphthyridine-3-carboxylic acid ethyl ester
1-(2,4-difluoro-phenyl)-6-fluoro-7-(4-{2-fluoro-4-[5-(R)-(1-hydroxy-prop-2-inyl)-2-oxo-oxazolidin-3-yl]-phenyl}-piperazin-1-yl)-4-oxo-1,4-dihydro-[1,8]naphthyridine-3-carboxylic acid
7-(4-{4-[5-(S)-(acetylamino-methyl)-2-oxo-oxazolidin-3-yl]-2-fluoro-phenyl}-piperazin-1-yl)-1-(2,4-difluoro-phenyl)-6-fluoro-4-oxo-1,4-dihydro-[1,8]naphthyridine-3-carboxylic acid
7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-[1,8]naphthyridine-3-carboxylic acid
7-(4-{4-[5-(S)-(acetylamino-methyl)-2-oxo-oxazolidin-3-yl]-2-fluoro-phenyl}-piperazin-1-yl)-6,8-difluoro-1-(2-fluoro-ethyl)-4-oxo-1,4-dihydro-quinoline-3-carboxylic acid
1-(2,4-Difluoro-phenyl)-6-fluoro-7-(4-{2-fluoro-4-[5-(S)-(isoxazol-3-ylaminomethyl)-2-oxo-oxazolidin-3-yl]-phenyl}-piperazin-1-yl)-4-oxo-1,4-dihydro-[1,8]naphthyridine-3-carboxylic acid
1-ethyl-6,8-difluoro-7-[4-(2-fluoro-4-{5-[(3-methyl-thioureido)-methyl]-2-oxo-oxazolidin-3-yl}-phenyl)-piperazin-1-yl]-4-oxo-1,4-dihydro-quinoline-3-carboxylic acid
1-cyclopropyl-6-fluoro-7-[4-(2-fluoro-4-{2-oxo-5-(S)-[(3-propyl-thioureido)-methyl]-oxazolidin-3-yl}-5 phenyl)-piperazin-1-yl]-4-oxo-1,4-dihydro-quinoline-3-carboxylic acid
1-cyclopropyl-6-fluoro-7-[4-{2-fluoro-4-[5-(S)-(methanesulfonylamino-methyl)-2-oxo-oxazolidin-3-yl]-phenyl}-piperazin-1-yl)-4-oxo-1,4-dihydro-quinoline-3-carboxylic acid
7-(4-{4-[5-(S)-(Acetylamino-methyl)-2-oxo-oxazolidin-3-yl]-phenyl}-piperazin-1-yl)-1-ethyl-6,8-fluoro-4-oxo-1,4-dihydro-quinoline-3-carboxylic acid ethyl ester
1-cyclopropyl-6-fluoro-7-[4-(2-fluoro-4-{2-oxo-5-(S)-[(2,2,2-trifluoro-acetylamino)-methyl]-oxazolidin-3-yl}-phenyl)-piperazin-1-yl]-4-oxo-1,4-dihydro-quinoline-3-carboxylic acid
7-(4-{4-[5-(S)-(benzoylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid
7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid methyl ester
7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid ethyl ester
7-(4-{4-[5-(S)-(acetylamino-methyl)-2-oxo-oxazolidin-3-yl]-2-fluoro-phenyl}-piperazin-1-yl)-1-ethyl-6,8-difluoro-4-oxo-1,4-dihydro-quinoline-3-carboxylic acid methyl ester
7-(4-{4-[5-(S)-(acetylamino-methyl)-2-oxo-oxazolidin-3-yl]-2-fluoro-phenyl}-piperazin-1-yl)-1-ethyl-6,8-difluoro-4-oxo-1,4-dihydro-quinoline-3-carboxylic acid ethyl ester
7-(4-{4-[5-(S)-(acetylamino-methyl)-2-oxo-oxazolidin-3-yl]-2-fluoro-phenyl}-piperazin-1-yl)-1-ethyl-6-fluoro-4-oxo-1,4-dihydro-quinoline-3-carboxylic acid methyl ester
7-(4-{4-[5-(S)-(acetylamino-methyl)-2-oxo-oxazolidin-3-yl]-2-fluoro-phenyl}-piperazin-1-yl)-1-ethyl-6-fluoro-4-oxo-1,4-dihydro-quinoline-3-carboxylic acid ethyl ester
9-(4-{4-[5-(S)-(acetylamino-methyl)-2-oxo-oxazolidin-3-yl]-phenyl}-piperazin-1-yl)-8-fluoro-3-methyl-6-oxo-2,3-dihydro-6H-1-oxa-3a-aza-phenalen-5-carboxylic acid methyl ester
9-(4-{4-[5-(S)-(acetylamino-methyl)-2-oxo-oxazolidin-3-yl]-phenyl}-piperazin-1-yl)-8-fluoro-3-methyl-6-oxo-2,3-dihydro-6H-1-oxa-3a-aza-phenalen-5-carboxylic acid ethyl ester
9-[3-({4-[5-(S)-(acetylamino-methyl)-2-oxo-oxazolidin-3-yl]-2-fluoro-phenyl}-methyl-amino) -pyrrolidin-1-yl]-8-fluoro-3-methyl-6-oxo-2,3-dihydro-6H-1-oxa-3a-aza-phenalen-5-carboxylic acid methyl ester
9-[3-({4-[5-(S)-(acetylamino-methyl)-2-oxo-oxazolidin-3-yl]-2-fluoro-phenyl}-methyl-amino)-pyrrolidin-1-yl]-8-fluoro-3-methyl-6-oxo-2,3-dihydro-6H-1-oxa-3a-aza-phenalen-5-carboxylic acid ethyl ester
9-(4-{4-[5-(S)-(acetylamino-methyl)-2-oxo-oxazolidin-3-yl]-2-fluoro-phenyl}-piperazin-1-yl)-8-fluoro-3-methyl-6-oxo-2,3-dihydro-6H-1-oxa-3a-aza-phenalen-5-carboxylic acid methyl ester
9-(4-{4-[5-(S)-(acetylamino-methyl)-2-oxo-oxazolidin-3-yl]-2-fluoro-phenyl}-piperazin-1-yl) -8-fluoro-3-methyl-6-oxo-2,3-dihydro-6H-1-oxa-3a-aza-phenalen-5-carboxylic acid ethyl ester
7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid methyl ester
7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic acid ethyl ester
1-cyclopropyl-6-fluoro-7-[4-(2-fluoro-4-{5-(S)-[(3-methyl-thioureido)-methyl]-2-oxo-oxazolidin-3-yl}-phenyl)-piperazin-1-yl]-4-oxo-1,4-dihydro-quinoline-3-carboxylic acid methyl ester
1-cyclopropyl-6-fluoro-7-[4-(2-fluoro-4-{5-(S)-[(3-methyl-thioureido)-methyl]-2-oxo-oxazolidin-3-yl}-phenyl)-piperazin-1-yl]-4-oxo-1,4-dihydro-quinoline-3-carboxylic acid ethyl ester
7-(4-{4-[5-(S)-(acetylamino-methyl)-2-oxo-oxazolidin-3-yl]-2-fluoro-phenyl}-piperazin-1-yl)-1-cyclopropyl-6-fluoro-4-oxo-1,4-dihydro-[1,8]naphthyridine-3-carboxylic acid methyl ester
7-(4-{4-[5-(S)-(acetylamino-methyl)-2-oxo-oxazolidin-3-yl]-2-fluoro-phenyl}-piperazin-1-yl)-6,8-difluoro-1-(2-fluoro-ethyl)-4-oxo-1,4-dihydro-quinoline-3-carboxylic acid methyl ester
1-Ethyl-6,8-difluoro-7-[4-(2-fluoro-4-{5-(S)-[(3-methyl-thioureido)-methyl]-2-oxo-oxazolidin-3-yl}-phenyl)-piperazin-1-yl]-4-oxo-1,4-dihydro-quinoline-3-carboxylic acid ethyl ester

9. Process for obtaining a compound of general formula (I), according to claim 1, characterised in that it comprises the reaction of a compound of general formula (II) with a compound of general formula (III): 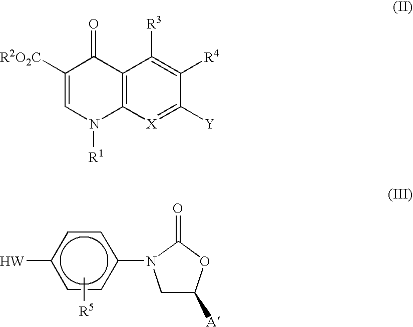
wherein:
A′ is:
a) —CH₂—NH—R⁷
b) —CHOH—C≡CH
c) 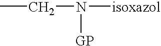
Y is an leaving group, such as an atom of halogen (F, Cl, Br, I), a tosilate or mesylate group, and the like;
R^{1.} R², R³, R⁴, R⁵, X and W have the meaning defined in claim 1;
GP is a protecting group of amines.

10. Process for obtaining a compound of general formula (I), according to claim 1, in which A is —CH₂—NH—R⁷ and R⁷is different from isoxazole, characterised in that it comprises the reaction of a compound of formula (V) 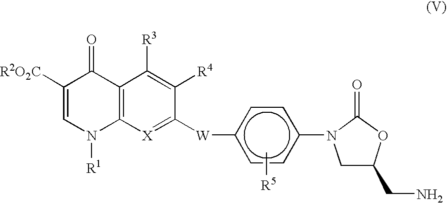
wherein R^{1.} R², R³, R⁴, R⁵, X and W have the meaning defined in claim 1.
with a compound of formula (VI) or with a compound of formula (VII)
R⁷—L (VI)
R⁸—N═C═Z (VII)
wherein L is a good leaving group, such as an atom of halogen (F, Cl, Br, I), a tosylate or mesylate group, and the like;
Z is Oxygen or Sulphur, and
R⁷ and R⁸ have the meaning defined in claim 1, with R⁷ being different from isoxazol.

11. Process for obtaining a compound of general formula (I), according to claim 1, in which A is —CH₂—NH—R⁷ and R⁷ is isoxazol, characterised in that it comprises the reaction of a compound of general formula (VIII): 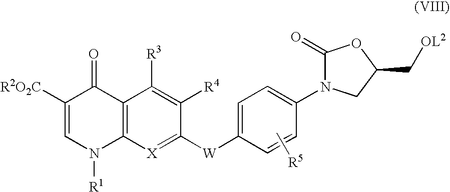
wherein
OL² represents a good leaving group, such as a residue of aryl or methyl sulphonic acid, substituted or not substituted, preferably by a tosylate or mesylate group;
R^{1.} R², R³, R⁴, R⁵, X and W have the meaning defined in claim 1;
with isaoxazolil-3-amine, the amine group being protected with a protecting group of amines.

12. Process for obtaining a compound of general formula (I), according to claim 1, in which R² is hydrogen, characterised in that it comprises the hydrolysis of a boron chelate of formula (IX) 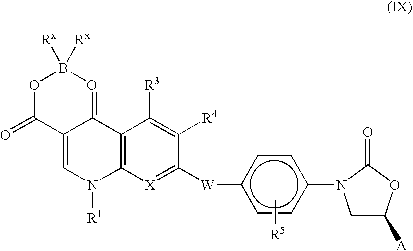
wherein
R^{X} can be F or CH₃COO—;
A, R^{1.} R³, R⁴, R⁵, X and W have the meaning defined in claim 1.

13. Process for obtaining a compound of general formula (I), according to claim 1, in which A is
—CHOH—C≡CH
characterised in that it comprises the reaction of a compound of formula (IV) 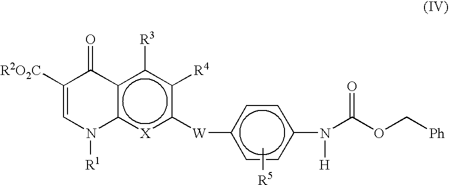
wherein R^{1.} R², R³, R⁴, R⁵, X and W have the meaning defined in claim 1,
with 2,3-hydroxy-pent-4-inyl p-toluenesulphonate.

14. Process as claimed in any of claims 11 to 13, characterised in that it comprises subjecting the product obtained, optionally, to one or more of the following final steps:
a) Conversion of a compound of general formula (I) into another compound of general formula (I);
b) Elimination of the protecting group;
c) Preparation of a pharmacologically acceptable salt of a compound of formula (I) and/or a pharmacologically acceptable solvate thereof.

15. Compound of formula (V) 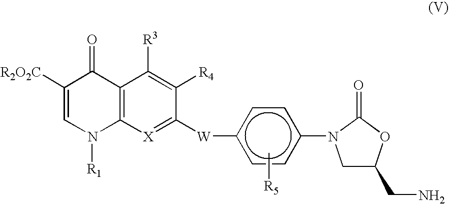
wherein R^{1.} R², R³, R⁴, R⁵, X and W have the meaning defined in claim 1.

16. Compound of formula (X) 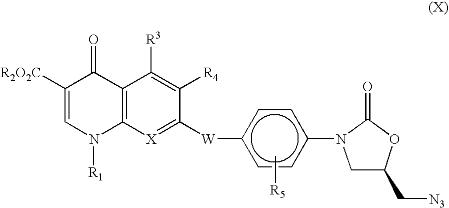
wherein R^{1.} R², R³, R⁴, R⁵, X and W have the meaning defined in claim 1.

17. Compound of formula (XI) 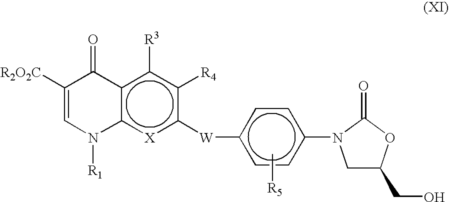
wherein R^{1.} R², R³, R⁴, R⁵, X and W have the meaning defined in claim 1.

18. Pharmaceutical composition which comprises a compound of general formula (I) according to any of claims 1 to 8, for use as a medicament.

19. Use of a compound of general formula (I), according to any of claims 1 to 8, for the preparation of a pharmaceutical composition for treating microbial infections in humans or warm-blooded animals.

20. Pharmaceutical composition which comprises a compound of general formula (I) according to any of claims 1 to 8 in a therapeutically active quantity and with a suitable quantity of at least one excipient.
